SEQUENCE LISTING (18 sequences)

SEQ ID NO: 1 - DNA, 1600 bp, Clostridium tetani
  CDS (88)..(1476)
ggaaacagctatgaccatgattacgccaagctcgaaattaaccctcactaaagggaacaaaagctggagctcggtacccgggccaccatggttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgagggatggacaaatgattaaacagattgatatgttcatgacatatgcccgggatcctctagagtcgacctcgagggggggcccggtacccaattcgccctatagtgagtcgtattacaattcactggccgtcgttttacaa

SEQ ID NO: 2 - protein, 463 aa, Clostridium tetani
MVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 3 - DNA, 1392 bp, Clostridium tetani
atggttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgagggatggacaaatgattaa

SEQ ID NO: 4 - DNA, 49 bp, Artificial Sequence
  Description of Artificial Sequence Primer
ccccccgggccaccatggttttttcaacaccaattccattttcttattc

SEQ ID NO: 5 - DNA, 18 bp, Artificial Sequence
  Description of Artificial Sequence Primer
ctaaaccagtaatttctg

SEQ ID NO: 6 - DNA, 25 bp, Artificial Sequence
  Description of Artificial Sequence Primer
aattatggactttaaaagattccgc

SEQ ID NO: 7 - DNA, 24 bp, Artificial Sequence
  Description of Artificial Sequence Primer
ggcattataacctactcttagaat

SEQ ID NO: 8 - DNA, 27 bp, Artificial Sequence
  Description of Artificial Sequence Primer
aatgcctttaataatcttgatagaaat

SEQ ID NO: 9 - DNA, 41 bp, Artificial Sequence
  Description of Artificial Sequence Primer
ccccccgggcatatgtcatgaacatatcaatctgtttaatc

SEQ ID NO: 10 - DNA, 24 bp, Artificial Sequence
  Description of Artificial Sequence Primer
ctgaatatcgacggtttccatatg

SEQ ID NO: 11 - DNA, 40 bp, Artificial Sequence
  Description of Artificial Sequence Primer
ggcagtctcgagtctagaccatggctttttgacaccagac

SEQ ID NO: 12 - DNA, 20 bp, Artificial Sequence
  Description of Artificial Sequence Synthetic oligonucleotide linker
catgactggggatccccagt

SEQ ID NO: 13 - DNA, 24 bp, Artificial Sequence
  Description of Artificial Sequence Primer
tatgataaaaatgcatctttagga

SEQ ID NO: 14 - DNA, 37 bp, Artificial Sequence
  Description of Artificial Sequence Primer
tggagtcgacgctagcaggatcatttgtccatccttc

SEQ ID NO: 15 - DNA, 8 bp, Artificial Sequence
  Description of Artificial Sequence Synthetic oligonucleotide linker
gctagcgc

SEQ ID NO: 16 - DNA, 17 bp, Artificial Sequence
  Description of Artificial Sequence Synthetic oligonucleotide linker
gatatcggcgcgccagc

SEQ ID NO: 17 - DNA, 17 bp, Artificial Sequence
  Description of Artificial Sequence Synthetic oligonucleotide linker
tggcgcgccgatatcgc

SEQ ID NO: 18 - DNA, 14 bp, Artificial Sequence
  Description of Artificial Sequence Synthetic oligonucleotide linker
tcgatggcgcgcca

(19) United States
(12) Patent Application Publication
(10) Pub. No.: US 2010/0081197 A1
(43) Pub. Date: Apr. 1, 2010

(54) In vivo modulation of neuronal transport

(76) Inventors: Sylvie Roux, Paris (FR); Philippe Brulet, Paris (FR); Cecile Saint Cloment, Paris (FR); Julien Barbier, Paris (FR); Jordi Molgo, Paris (FR)

Correspondence Address: Finnegan Henderson Farabow Garrett & Dunner, LLP, 901 New York Avenue, N.W., Washington, DC, 20001--441, US

(21) Appl. No.: 11/898,789

(22) Filed: Sep. 14, 2007

Related U.S. Application Data
(63) Continuation-in-part of application No. 10/662,808, filed on Sep. 16, 2003.
Application No. 10/662,808 is a continuation of application No. 09/816,467, filed on Mar. 26, 2001, now Pat. No. 7,435,792.
Application No. 09/816,467 is a continuation of application No. 09/129,368, filed on Aug. 5, 1998, now abandoned.
(60) Provisional application No. 60/055,615, filed on Aug. 14, 1997.
(60) Provisional application No. 60/065,236, filed on Nov. 13, 1997.

(51) Int. Cl. C12N 5/00

(52) U.S. Cl. 435/375

(57) ABSTRACT

A method for visualizing an active synapse wherein said method comprises: (a) exposing cells forming the active synapse to a biomarker comprising at least fragment C of tetanus toxin and a reporter protein; and (b) visualizing the biomarker; wherein the accumulation of the biomarker into dendritic spines of the cells allows visualization of an active synapse. Also, a method for screening molecules capable of modulating synapse activity is provided. A kit useful for the early diagnosis of neurodegenerative disease comprises a biomarker comprising at least fragment C of tetanus toxin and a reporter protein.

### CROSS REFERENCE TO RELATED APPLICATIONS

The present application is a continuation-in-part of U.S. application Ser. No. 10/662,808, filed Sep. 16, 2003, which is a continuation of U.S. application Ser. No. 09/816,467, filed Mar. 26, 2001, which is a continuation of U.S. application Ser. No. 09/129,368, filed Aug. 5, 1998, now abandoned, which claims the benefit of Provisional Application No. 60/055,615, filed Aug. 14, 1997 and Provisional Application No. 60/065,236, filed Nov. 13, 1997. The entire disclosure of each of these applications is relied upon and incorporated by reference herein.

### BACKGROUND OF THE INVENTION

This invention relates to the use of part of tetanus toxin for delivering a composition to the central nervous system of a human or animal. This invention also relates to a hybrid fragment of tetanus toxin, a polynucleotide that hybridizes with natural tetanus toxin, and a composition containing the tetanus toxin fragment as an active molecule. Further, this invention relates to a vector comprising a promoter and a nucleic acid sequence encoding the tetanus toxin fragment.

Tetanus toxin is produced by Clostridium tetani as an inactive, single, polypeptide chain of 150 kD composed of three 50 kD domains connected by protease-sensitive loops. The toxin is activated upon selective proteolytic cleavage, which generates two disulfide-linked chains: L (light, 50 kD) and H (heavy, 100 kD) [Montecucco C. and Schiavo G. Q. Rev. Biophys., (1995), 28: 423-472].

Evidence for the retrograde axonal transport of tetanus toxin to central nervous system (CNS) has been described by Erdmann et al. [Naunyn Schmiedebergs Arch Phamacol., (1975), 290:357-373], Price et al. [Science, (1975), 188:945-94], and Stoeckel et al. [Brain Res., (1975), 99:1-16]. In each of these studies, radiolabeled toxin was found inside membrane bound vesicles. Another property was the transynaptic movement of tetanus toxin that was demonstrated first by autoradiographic localization of ¹²⁵ I-labeled tetanus toxin in spinal cord interneurons after injection into a muscle [Schwab and Thoenen, Brain Res., (1976), 105:218-227].

The structure of this tetanus toxin has been elucidated by Helting et al. [J. Biol. Chem., (1977), 252:187-193]. Papain cleaves the tetanus toxin in two fragments:

the C terminal part of the heavy chain, 451 amino acids, also called fragment C; and

the other part contained the complementary portion called fragment B linked to the light chain (fragment A) via a disulfide bond.

European Patent No. EP 0 030 496 B1 showed the retrograde transport of a fragment B-II_{b} to the CNS and was detected after injection in the median muscle of the eye in primary and second order neurons. This fragment may consist of “isofragments” obtained by clostridial proteolysis. Later, this fragment B-II_{b} was demonstrated to be identical to fragment C obtained by papain digestion by Eisel et al. [EMBO J., 1986, 5:2495-2502].

This EP patent also demonstrated the retrograde transport of a conjugate consisting of a I_{bc} tetanus toxin fragment coupled by a disulfide bond to B-II_{b} from axonal endings within the muscle to the motoneuronal perikarya and pericellular spaces. (The I_{bc} fragment corresponds to the other part obtained by papain digestion as described above by Helting et al.). There is no evidence that this conjugate was found in second order neurons. The authors indicated that a conjugate consisting of the fragment B-II_{b} coupled by a disulfide bond to a therapeutic agent was capable of specific fixation to gangliosides and synaptic membranes. No result showed any retrograde axonal transport or a transynaptic transport for such conjugate.

Another European Patent, No. EP 0 057 140 B1, showed equally the retrograde transport of a fragment II_{c} to the CNS. As in the European Patent No. EP 0 030 496 B1, the authors indicated that a conjugate consisting of the fragment II_{c} and a therapeutic agent was capable of specific fixation, but no result illustrated such allegation. This fragment II_{c} corresponds to the now called fragment C obtained by papain digestion.

Francis et al. [J. Biol. Chem., (1995), 270(25)15434-15442] led an in vitro study showing the internalization by neurons of hybrid between SOD-1 (Cu Zn superoxide dismutase) and a recombinant C tetanus toxin fragment by genetic recombination. This recombinant C tetanus toxin fragment was obtained from Halpern group. (See ref. 11).

Moreover, Kuypers H. G. J. M and Ugolini G. [TINS, (1990), 13(2):71-75] indicated in their publication concerning viruses as transneuronal tracers that, despite the fact that tetanus toxin fragment binds to specific receptors on neuronal membranes, transneuronal labeling is relatively weak and can be detected only in some of the synaptically connected neurons.

Notwithstanding these advances in the art, there still exists a need for methods for delivering compositions into the human or animal central nervous system. There also exists a need in the art for biological agents that can achieve this result.

Additionally, activity-dependent modification of neuronal connectivity and synaptic plasticity play an important role in the development and function of the nervous system. Recently, much effort has been dedicated to following such modifications by the engineering of new optically detectable genetic tools. For example, fused to a reporter gene such as LacZ or GFP (Green Fluorescent Protein), the atoxic C-terminal fragment of tetanus toxin (or TTC fragment) can traffic retrogradely and transsynaptically inside a restricted neural network either after direct injection of the hybrid protein (Coen et al., 1997), or when expressed as a transgene in mice (Maskos et al., 2002). The dynamics of βgal-TTC clustering at the neuromuscular junction (NMJ) is strongly dependent on a presynaptic neuronal activity and probably involves fast endocytic pathways (Miana-Mena et al., 2002). Neuronal activity may induce this clustering and internalization at the NMJ by enhancing the secretion and/or action of various molecules at the synapse.

Over the past decade, various data indicate that neurotrophins, a family of structurally and functionally related proteins, including NGF (Nerve Growth Factor); BDNF (Brain Derived Neurotrophic Factor); Neurotrophin 3 (NT-3) and Neurotrophin 4 (NT-4), not only promote neuronal survival and morphological differentiation, but also can acutely modify synaptic transmission and connectivity in central synapses, thus providing a connection between neuronal activity and synaptic plasticity (McAllister et al., 1999; Poo, 2001; Tao and Poo, 2001). The role of these factors in neurotransmission between motoneurons and skeletal muscle cells has been studied using Xenopus nerve-muscle co-culture studies, whereby the treatment of these cultures with exogenous BDNF, NT-3 or NT-4 leads to an increase of synaptic transmission by enhancing neurotransmitter secretion (Lohof et al., 1993; Stoop and Poo, 1996; Wang and Poo, 1997). Moreover, the muscular expression of NT-3 and NT-4 (Funakoshi et al., 1995; Xie et al., 1997), as well as NT-4 secretion (Wang and Poo, 1997) are regulated by electrical activity. This family of proteins thus provides a molecular link between electrical neuronal activity and synaptic changes.

The cellular actions of neurotrophins are mediated by two types of receptors: the p75^{NTR} receptor, mainly expressed during early neuronal development, and a Trk tyrosine kinase receptor (Bothwell, 1995). The interaction of neurotrophins with Trk receptors is specific. TrkB and TrkC, are activated by BDNF/NT-4 and NT-3, respectively, and are expressed by motor neurons. TrkA, which is expressed by sensory neurons, is activated by NGF. Recently, evidence for a co-trafficking between TIC and the neurotrophin receptor p75^{NTR} has been reported in cultured motoneurons (Lalli and Schiavo, 2002), as well as the activation by tetanus toxin and the TTC fragment of intracellular pathways involving Trk receptors in cultured cortical neurons (Gil et al., 2003).

Notwithstanding the knowledge in the art, there still exists a need for understanding the influences of neurotrophins and other neurotrophic factors on TTC traffic at the NMJ in vivo and for developing methods of using these neurotrophins and neurotrophic factors, and agonists or antagonists thereof, to modulate the neuronal transport of a tetanus toxin or a fusion protein comprising a fragment C of the tetanus toxin.

### SUMMARY OF THE INVENTION

This invention aids in fulfilling these needs in the art. More particularly, this invention provides a method for in vivo delivery of desired composition into the central nervous system (CNS) of the mammal, wherein the composition comprises a non-toxic proteolytic fragment of tetanus toxin (TT) in association with at least a molecule having a biological function. The composition is capable of in vivo retrograde transport and transynaptic transport into the CNS and of being delivered to different areas of the CNS.

This invention also provides a hybrid fragment of tetanus toxin comprising fragment C and fragment B or a fraction thereof of at least 11 amino acid residues or a hybrid fragment of tetanus toxin comprising fragment C and fragment B or a fraction thereof of at least 11 amino acid residues and a fraction of fragment A devoid of its toxic activity corresponding to the proteolytic domain having a Zinc-binding motif located in the central part of the chain between the amino acids 225 and 245, capable of transferring in vivo a protein, a peptide, or a polynucleotide through a neuromuscular junction and at least one synapse.

Further, this invention provides a composition comprising an active molecule in association with the hybrid fragment of tetanus toxin (TT) or a variant thereof. The composition is useful for the treatment of a patient or an animal affected with CNS disease, which comprises delivering a composition of the invention to the patient or animal. In addition, the composition of this invention may be useful to elicit an immune response in the patient or animal affected with CNS, which comprises delivering a composition of the invention to the patient or animal.

Moreover, this invention provides polynucleotide variant fragments capable of hybridizing under stringent conditions with the natural tetanus toxin sequence. The stringent conditions are for example as follows: at 42.0 for 4 to 6 hours in the presence of 6×SSC buffer, 1×Denhardt's Solution, 1% SDS, and 250 μg/ml of tRNA. (1×SSC corresponds to 0.15 M NaCl and 0.05 M sodium citrate; 1×Denhardt's solution corresponds to 0.02% Ficoll, 0.02% polyvinyl pyrrolidone and 0.02% bovine serum albumin). The two wash steps are performed at room temperature in the presence of 0.1×SCC and 0.1% SDS.

A polynucleotide variant fragment means a polynucleotide encoding for a tetanus toxin sequence derived from the native tetanus toxin sequence and having the same properties of transport.

In addition, the invention provides a vector comprising a promoter capable of expression in muscle cells and optionally an enhancer, a nucleic acid sequence coding for the fragment of tetanus toxin of the invention or an amino acid variant fragment of the invention associated with a polynucleotide coding for a protein or a polypeptide of interest. In a preferred embodiment of the invention the promoter can be the CMV promoter and preferably the CMV promoter contained in pcDNA 3.1 (In Vitrogen, ref. V790-20), or the promoter β actin as described in Bronson S. V. et al. (PNAS, 1996, 93:9067-9072).

In addition, the invention provides a vector comprising a promoter capable of expression in neuronal cells or in precursors (such NT2(hNT) precursor cells from Stratagene reference #204101) and optionally an enhancer, a nucleic acid sequence coding for the fragment of tetanus toxin of the invention or an amino acid variant fragment of the invention associated with a polynucleotide coding for a protein or a polypeptide of interest. In a preferred embodiment of the invention the promoter can be β actin (see the above reference). These vectors are useful for the treatment of a patient or an animal infected with CNS disease comprising delivering the vector of the invention to the patient or animal. In addition, these vectors are useful for eliciting immune responses in the patient or animal.

One advantage of the present invention comprising the fragment of tetanus toxin (fragment A, B, and C) is to obtain a better transport of the fragment inside the organism compared with fragment C. Another advantage of the composition of the invention is to obtain a well defined amino acid sequence and not a multimeric composition. Thus, one can easily manipulate this composition in gene therapy.

In another embodiment, this invention provides a method of modulating the transport in a neuron of a neurotoxin, such as the tetanus toxin, or a fusion protein comprising a fragment C of the tetanus toxin. These methods comprise administering neurotrophic factors such as BDNF, NT-4, and GDNF, and agonists and antagonists thereof, to modulate internalization at a neuromuscular junction of a neurotoxin or a fusion protein comprising the TTC fragment according to the invention.

In one embodiment, these methods further comprise administering to the neuron a TrkB receptor agonist or a TrkB receptor antagonist in an amount sufficient to modulate the neuronal transport of the tetanus toxin or the fusion protein. The term “modulate” and its cognates refer to the capability of a compound acting as either an agonist or an antagonist of a certain reaction or activity. The term modulate, therefore, encompasses the terms “increase” and “decrease.” The term “increase,” for example, refers to an increase in the neuronal transport of a polypeptide in the presence of a modulatory compound, relative to the transport of the polypeptide in the absence of the same compound. Analogously, the term “decrease” refers to a decrease in the neuronal transport of a polypeptide in the presence of a modulatory compound, relative to the transport of the polypeptide in the absence of the same compound. The neuronal transport of polypeptides can be measured as described herein or by techniques generally known in the art.

The TrkB receptor agonists include neurotrophic factors that activate a TrkB receptor, such as a Brain Derived Neurotrophic Factor or a Neurotrophin 4. The TrkB receptor agonists can also include antibodies that bind to TrkB receptors and activate them. These methods of using TrkB receptor agonists provide useful methods for enhancing the neuronal transport of a tetanus toxin or a tetanus toxin fusion protein.

The TrkB receptor antagonists include antibodies that bind to a TrkB receptor agonist, such as those described above, and thereby decrease the activation of a TrkB receptor. For example, these antibodies can be directed to neurotrophic factors that activate a TrkB receptor, such as a Brain Derived Neurotrophic Factor or a Neurotrophin 4. In addition, TrkB receptor antagonists include antibodies that bind to TrkB receptors and inactivate them. These methods of using TrkB receptor agonists provide useful methods for decreasing or preventing the neuronal transport of a tetanus toxin or a tetanus toxin fusion protein.

In another embodiment, these methods further comprise administering to the neuron a GFRα/cRET receptor agonist or a GFRα/cRET receptor antagonist in an amount sufficient to modulate the neuronal transport of the tetanus toxin or the fusion protein.

The GFRα/cRET receptor agonists include neurotrophic factors that activate a GFRα/cRET receptor, such as a Glial-Derived Neurotrophic Factor. The GFRα/cRET receptor agonists can also include antibodies that bind to GFRα/cRET receptors and activate them. These methods of using GFRα/cRET receptor agonists provide useful methods for enhancing the neuronal transport of a tetanus toxin or a tetanus toxin fusion protein.

The GFRα/cRET receptor antagonists include antibodies that bind to a GFRα/cRET receptor agonist, such as those described above, and thereby decrease the activation of a GFRα/cRET receptor. For example, these antibodies can be directed to neurotrophic factors that activate a GFRα/cRET receptor, such as a Glial-Derived Neurotrophic Factor. In addition, GFRα/cRET receptor antagonists include antibodies that bind to GFRα/cRET receptors and inactivate them. These methods of using GFRα/cRET receptor agonists provide useful methods for decreasing or preventing the neuronal transport of a tetanus toxin or a tetanus toxin fusion protein.

In these methods, the agonist or antagonist can be administered to neuronal cells that already contain a tetanus toxin or a fusion protein. Alternatively, the tetanus toxin or fusion protein can be administered concurrently with or after the administration of the agonist or antagonist.

In one embodiment, the TTC-containing fusion proteins of the present invention comprises a second protein that is encoded by a reporter gene, such as the lac Z gene or the Green Fluorescent Protein gene. Such fusion proteins are useful for visualizing modulation of the synaptic plasticity in vivo, including in a human, for example by magnetic resonance imaging. For example, the fusion proteins can be used to monitor and detect the effects of a compound, such as a neurotrophic factor, on neuronal transport. In these methods, the compound and the fusion protein are administered to a neuron, and the fusion protein is detected to determine the effect of the compound on the neuronal transport. In addition, the fusion proteins can be used to detect modifications in trafficking patterns within a restricted neural network, such as those used in known animal models for neurodegenerative diseases. The fusion proteins can also be used in screening methods to detect compounds that reduce or prevent neuronal transport of a tetanus toxin. Compounds so identified can be used to prevent or treat tetanus infections.

The TTC fragment can also be coupled to a neurotrophic factor and administered to a patient to treat CNS pathologies associated with production defects of different factors. The TTC fragment could also be used as a vector for modulating interactions with proteins involved in neurodegenerative diseases.

The present invention also provides compositions comprising a TrkB receptor agonist or a GFRα/cRET receptor agonist and a fusion protein comprising a fragment C of the tetanus toxin fused to a second protein. In one embodiment, the TrkB agonist is a neurotrophic factor such as a Brain Derived Neurotrophic Factor or a Neurotrophin 4. In another embodiment, the GFRα/cRET receptor agonist is a neurotrophic factor, such as Glial-Derived Neurotrophic Factor.

### BRIEF DESCRIPTION OF THE DRAWINGS

The patent or application file contains at least one drawing executed in color. Copies of the patent or patent application publication with color drawing(s) will be provided by the Office upon request and payment of the necessary fee.

This invention will be more fully described with reference to the drawings in which:

FIG. 1 shows the DNA sequence and amino acid sequence of the TTC fragment cloned in pBS:TTC.

FIG. 2 shows the details of construct pBS:TTC.

FIG. 3 depicts pGEX:lacZ-TTC construct.

FIG. 4 shows construct pGEX:TTC-lacZ.

FIG. 5 depicts the details of the construct pCMV:lacZ-TTC.

FIG. 6 shows the confocal immunofluorescence analysis of GFP-TTC membrane traffic at mature mouse LAL NMJs. (A) Two hours after the subcutaneous injection of GFP-TTC in the vicinity of the LAL muscle, the probe (green) was concentrated at motor nerve endings of NMJ. Associated intramuscular motor axons were immunostained (red) with an antibody against NF200. GFP-TTC labeling was also detected in sensory nerve fibers (arrows) and at the nodes of Ranvier of myelinated axons (arrowheads). (B) Strong nodal labeling with GFP-TTC (green) (arrow) in a single living myelinated axon. Myelin was passively stained with RH414 dye (red). (C) Two hours after injection as in A, LAL muscle fibers were fixed and labeled for troponin T by indirect immunofluorescence. (C' and C″) Inset is a side view image of a NMJ showing that GFP-TTC staining (green) is located presynaptically. (D-G) LAL was harvested at various times after GFP-TTC injection and NMJ identified in red by labeling with TRITC-a-BTX (D′-G′). D-D′: 5 min; E-E′: 30 min; F-F′: 2 h and G-G′: 24 h. Scale bars: A, 20 μm; B, 8 μm; C: 20 λm; D, 2 μm; E-G, 5 μm.

FIG. 7 shows that BDNF increases GFP-TTC recruitment at the NMJ in a dose-dependent manner. (A) The NMJ on LAL muscles was identified by TRITC-a-BTX labeling 30 min after in vivo co-injection of GFP-TTC with various amounts of BDNF. The level of GFP fluorescence was quantified over these areas (see B). An enhancement of axonal labeling (arrows), more pronounced with higher BDNF concentration, was also detected. Scale bars: 20 μm. (B) Confocal sections of the NMJ were collected for analysis and projections generated. TRIT-a-BTX labeling determines the area of the NMJ over which the global GFP fluorescent signal was measured. For each, (n=15-20), the GFP fluorescence was divided by NMJ area (in μm²) to obtain the fluorescence level. Error bars indicate S.D. **P<0.005; t-test, vs control.

FIG. 8 depicts the immunofluorescence visualization of TrkB at the LAL NMJ. Two hours after GFP-TTC injection in LAL, confocal analysis was performed.

The fusion protein was identified in green directly by GFP fluorescence. TrkB, identified (in red) by indirect immunofluorescence (see material and methods), was located at the NMJ. However, when the two projections were overlaid, no overlap was found between the TrkB and the GFP-TTC signals. Scale bar: Top: 5 μm; Bottom: 2 μm.

FIG. 9 represents the results of experiments elucidating the mechanisms of GFP-TTC recruitment to the NMJ. (A) Quantification of GFP-TTC fluorescence was performed, as described in FIG. 7 at various time after co-injection with or without 50 ng BDNF. (B) After in vitro loading for 45 min with GFP-TTC, the excised LAL muscle was fixed and SV2 protein detected by indirect immunofluorescence (red). SV2 labeling was mostly diffuse and concentrated in a few areas of the NMJ (arrows). Co-localization of SV2 with GFP-TTC staining was only observed in a very limited number of areas. Scale bar: 8 μm. (C) Treatment with botulinum type-A neurotoxin to block synaptic vesicles exocytosis and endocytosis. 48 hours after BoTx/A injection (as described in material and methods), GFP-TTC, associated or not with 50 ng BDNF, was injected in LAL muscle and GFP fluorescence quantified as previously. **P<0.005; t-test, vs control; * P<0.005; t-test vs BoTx/A treatment. (D) Comparison of KCl induced depolarization and BDNF effects on GFP-TIC localization at the NMJ.

FIG. 10 depicts the localization of GFP-TTC probe in lipid microdomains. (A) 2 hours after intramuscular injection, GFP-TTC was found in detergent resistant membranes (DRMs) (lanes 4-6) isolated from gastrocnemius muscle, which also contained the raft marker caveolin-3. A small amount of GFP-TTC was also detected in the soluble fraction (lane 12). (B) GFP-TTC co-localized with the raft marker GM1 at the NMJ. NMJ were identified by Alexa 647-a-BTX binding (in blue). Whereas GFP-TTC was detected in less than 5 min at the NMJ, CT-b requires 3-5 hours. At this time, a diffuse staining which co-localized with the similar GFP-TTC labeling, was obtained, while a few patches labeling only for CT-b were also observed. (C) Intensity profiles of GFP-TTC (green) and Alexa 594 labeled-CT-b (red) were performed 5 or 24 h after intramuscular co-injection of both probes in gastrocnemius. Scale bar: 5 μm.

FIG. 11 shows a comparison of GFP-TTC and CT-b localization in motoneuron cell bodies. Twenty four hours after β-gal-TTC (A) or GFP-TTC and CT-b (B-E) intramuscular injection in gastrocnemius muscle, mice were perfused intracardially and their spinal cords removed. (A) X-gal reaction on spinal cord transerve sections showed labeling in motoneuron cell bodies but also in neurites (inset). (B) GFP-TTC and CT-b were detected on longitudinal section of spinal cord in a significant number of motoneurons. (C) Probes were detected in vesicles highly concentrated in cell bodies but also in neurites. (D) In neuronal extensions, GFP-TTC and CT-b were detected in different vesicular-like structures. Note that only few of them were positive for both probes. (E) Note that neither GFP-TTC nor CT-b were detected in the nucleus as shown in one optical section. Scale bars: A, 0.2 mm; inset, 50 μm; B, 20 μm; C, 10 μm; D, 5 μm; E, 2 μm.

FIG. 12. Confocal microscopic image of a whole-mount LAL neuromuscular preparation from a mouse injected with GFP-TTC. Two hours after the s.c. injection of GFP-TTC in the vicinity of the LAL muscle, the mouse was transcardially fixed, the LAL muscle was excised, and associated intramuscular motor axons were immunostained (red) with a monoclonal anti-neurofilament antibody (NF200). Note that the GFP-TTC probe (stained green) was concentrated at motor nerve endings of the NMJ. Scale bar, 20

FIG. 13. Brain-derived neurotrophic factor (BDNF) increases GFP-TTC recruitment and the kinetics of its internalization by nerve terminals of the NMJ. (A) Confocal microscopic projections of NMJs of LAL muscles stained by Alexafluor 594-conjugated-α-BTX labeling 30 min after injecting mice s.c. with GFP-TTC (control), and after injecting GFP-TTC s.c. with various amounts of BDNF. Note the enhancement of axonal labeling elicited by the highest BDNF concentrations tested (arrows). Scale bars, 20 μm. (B) Quantification of GFP-TTC fluorescence under control conditions and after co-injecting GFP-TTC and BDNF. Confocal images of microscopic projections from a series of optical sections were analyzed, as described in Example 15, in order to determine the standardized amount of GFP-TTC per NMJ. Each column represents the mean values +/− SEM of 15-20 NMJs examined in three independent experiments. Note the significant increase in GFP-TTC localization after its co-injection with BDNF. **P<0.005; t-test, vs. control. (C) Time-dependence of GFP-TTC internalization by motor nerve terminals under control conditions (blue column) and after co-injecting BDNF (50 ng) and GFP-TTC (red column). Each column represents the mean of triplicate experiments +/− SEM. *P<0.05; **P<0.005.

FIG. 14. Endogenous BDNF is involved in GFP-TTC internalization. Pretreatment of mice with BDNF-neutralizing antibody 30 min before injecting them with GFP-TTC, or before co-injecting them with BDNF and GFP-TTC, significantly reduced (compared with mice pretreated with a control IgY antibody preparation) GFP-TTC's localization at the NMJ. The amount of GFP-TTC at the NMJ was quantified as outlined in FIG. 13. Each column represents the mean of triplicate experiments **P<0.005; t-test, vs. control.

FIG. 15. Visualization of TrkB at the LAL NMJ. LAL muscles were examined by confocal microscopy 30 min post-injection of 50 μg of GFP-TTC. The fusion protein was identified by the fluorescence of GFP (green); TrkB was localized similarly as GFP-TTC, mainly in the presynaptic side of the NMJ (compared TrkB and α-BTX labeling). However, no overlapping was observed between TrkB and GFP-TTC signals. Scale bar, 5 μm,

FIG. 16. Schematic of the procedure used to ligate the sciatic nerve.

### DETAILED DESCRIPTION

Tetanus toxin is a potent neurotoxin of 1315 amino acids that is produced by Clostridium tetani (1, 2). It prevents the inhibitory neurotransmitter release from spinal cord interneurons by a specific mechanism of cell intoxication (for review see ref 3). This pathological mechanism has been demonstrated to involve retrograde axonal and transynaptic transport of the tetanus toxin. The toxin is taken up by nerve endings at the neuromuscular junction, but does not act at this site; rather, the toxin is transported into a vesicular compartment and travels along motor axons for a considerable distance until it reaches its targets. The transynaptic movement of tetanus toxin was first demonstrated by autoradiographic localization in spinal cord interneurons after injection into a muscle (4). However, previous studies of transynaptic passage of tetanus toxin from motoneurons were limited by the rapid development of clinical tetanus and death of the experimental animal (4, 5, 6).

A fragment of tetanus toxin obtained by protease digestion, the C fragment, has been shown to be transported by neurons in a similar manner to that of the native toxin without causing clinical symptoms (7, 8, 9, 10). A recombinant C fragment was reported to possess the same properties as the fragment obtained by protease digestion (11). The fact that an atoxic fragment of the toxin molecule was able to migrate retrogradely within the axons and to accumulate into the CNS led to speculation that such a fragment could be used as a neurotrophic carrier (12). A C fragment chemically conjugated to various large proteins was taken up by neurons in tissue culture (13) and by motor neurons in animal models (ref. 12, 14, and 15). According to the invention the fragment of tetanus toxin consists of a non-toxic proteolytic fragment of tetanus toxin (TT) comprising a fragment C and a fragment B or a fraction thereof of at least 11 amino acid residues or a non-toxic proteolytic fragment of tetanus toxin (TT) comprising a fragment C and a fragment B or a fraction thereof of at least 11 amino acids residues and a fraction of a fragment A devoid of its toxic activity corresponding to the proteolytic domain having a zinc-binding motif located in the central part of the chain between the amino acids 225 and 245 (cf. Montecucco C. and Schiavo G. Q. Rev. Biophys., (1995), 28:423-472). Thus the fraction of the fragment A comprises, for example, the amino acid sequence 1 to 225 or the amino acid sequence 245 to 457, or the amino acid sequence 1 to 225 associated with amino acid sequence 245 to 457.

The molecule having a biological function is selected from the group consisting of protein of interest, for example, for the compensation or the modulation of the functions under the control of the CNS or the spinal cord or the modulation of the functions in the CNS or the spinal cord, or protein of interest to be delivered by gene therapy expression system to the CNS or the spinal cord. The proteins of interest are, for example, the protein SMN implicated in spinal muscular atrophy (Lefebvre et al., Cell, (1995), 80:155-165 and Roy et al., Cell, (1955), 80:167-178); neurotrophic factors, such as BDNF (Brain-derived neurotrophic factor); NT-3 (Neurotrophin-3); NT-4/5; GDNF (Glial cell-line-derived neurotrophic factor); IGF (Insulin-like growth factor) (Oppenheim, Neuron, (1996), 17:195-197; Thoenen et al., Exp. Neurol., (1933), 124:47-55 and Henderson et al., Adv. Neurol., (1995), 68:235-240); or PNI (protease nexin I) promoting neurite outgrowth (this factor can be used for the treatment of Alzheimer disease (Houenou et al., PNAS, (1995), 92:895-899)); or SPI3 a serine protease inhibitor protein (Safaei, Dev. Brain Res., (1997), 100: 5-12); or ICE (Interleukin-1β converting Enzyme) a factor implicated in apoptosis, to avoid apoptosis (Nagata, Cell, (1997), 88:355-365); or BcI-2, a key intracellular regulator of programmed cell death (Jacobson, M. D. (1997), Current Biology, 7:R277-R281); or green fluorescent protein (Lang et al., Neuron, (1997), 18:857-863) as a marker; enzyme (ex: 13-Gal); endonuclease like I-Scel (Choulika A., et al. (1995), Molecular and Cellular biology, 15 (4):1968-1973 or CRE (Gu H., et al. (1994), Science, 265:103-106); specific antibodies; drugs specifically directed against neurodegenerative diseases such as latero spinal amyotrophy. Several molecules can be associated with a TT fragment.

In association means an association obtained by genetic recombination. This association can be realized upstream as well as downstream to the TT fragment. The preferred mode of realization of the invention is upstream and is described in detail; a downstream realization is also contemplated. (Despite Halpern et al., J. Biol. Chem., (1993), 268(15):11188-11192, who indicated that the carboxyl-terminal amino acids contain the domain required for binding to purified gangliosides and neuronal cells.)

The desired CNS area means, for example, the tongue which is chosen to direct the transport to hypoglossal motoneuron; the arm muscle which is chosen to direct the transport to the spinal cord motoneurons.

For this realization of transplantation of a neuron to the CNS or the spinal cord see Gage, F. H. et al. (1987), Neuroscience, 23:725-807, “Grafting genetically modified cells to the brain: possibilities for the future.”

The techniques for introducing the polynucleotides to cells are described in U.S. Pat. Nos. 5,580,859 and 5,589,466, which is relied upon and incorporated by reference herein. For example, the nucleotides may be introduced by transfection in vitro before reimplantation in area of the CNS or the spinal cord.

A chemical linkage is considered for a particular embodiment of the invention and comprises the association between the TT fragment and a polynucleotide encoding the molecule of interest with its regulatory elements, such as promoter and enhancer capable of expressing said polynucleotide. Then the TT fragment allows the retrograde axonal transport and/or the transynaptic transport, and the product of the polynucleotide is expressed directly in the neurons. This chemical linkage can be covalent or not, but preferably covalent performed by thiolation reaction or by any other binding reaction as described in “Bioconjugate Techniques” from Gret T. Hermanson (Academic press, 1996).

The axonal retrograde transport begins at the muscle level, where the composition of interest is taken up at the neuromuscular junction, and migrates to the neuronal body of the motoneurons (which are also called the first order neurons) in the CNS or spinal cord. First order neurons mean neurons that have internalized the composition of interest, and thus in this case, correspond to motoneurons.

The transynaptic retrograde transport corresponds to interneuron communications via the synapses from the motoneurons, and comprises second order neurons and higher order neurons (fourth order corresponding to neurons in the cerebral cortex).

The different stages of the neuronal transport are through the neuromuscular junction, the motoneuron, also called first order neuron, the synapse at any stage between the neurons of different order, neuron of order second to fourth order, which corresponds to the cerebral cortex.

In one embodiment of this invention, it is shown that a β-gal-TTC (TT-fragment C) hybrid protein retains the biological activities of both proteins in vivo. Therefore, the hybrid protein can undergo retrograde and transneuronal transport through a chain of interconnected neurons, as traced by its enzymatic activity. These results are consistent with those of others who used chemically conjugated TTC, or TTC fused to other proteins (12, 13, 14, 15). In these in vitro analyses, the activity of the conjugated or hybrid proteins was likewise retained or only weakly diminished. Depending on the nature of the TTC fusion partner, different types of potential applications can be envisioned. For example, this application can be used to deliver a biologically active protein into the CNS for therapeutic purposes. Such hybrid genes can also be used to analyze and map synaptically connected neurons if reporters, such as lacZ or the green fluorescent protein (GFP; 29) gene, were fused to TTC.

The retrograde transport of the hybrid protein may be demonstrated as follows. When injected into a muscle, β-gal activity rapidly localized to the somata of motoneurons that innervate the muscle. The arborization of the whole nerve, axon, somata and dendrites can easily be visualized. However, in comparison to the neurotropic viruses, the extent of retrograde transneuronal transport of the hybrid protein from the hypoglossal neurons indicates that only a subset of interconnected neurons is detected, although most areas containing second-order interneurons have been identified by the β-gal-TTC marker. Transneuronal uptake is mostly restricted to second order neurons. In such experiments, when a limited amount of a neuronal tracer is injected into a muscle or cell, only a fraction will be transported through a synapse, thereby imposing an experimental constraint on its detection. Presently, the most efficient method, in terms of the extent of transport, relies on neurotropic viruses. Examples include: alpha-herpes viruses, such as herpes simplex type 1 (HSV-1), pseudorabies virus (PrV), and rhabdoviruses (24, 25). Viral methods are very sensitive because each time a virus infects a new cell, it replicates, thereby amplifying the signal and permitting visualization of higher order neurons in a chain. Ultimately, however, one wants to map a neuronal network in an in vivo situation such as a transgenic animal. Here, the disadvantage of viral labeling is its potential toxicity. Most viruses are not innocuous for the neural cell, and their replication induces a cellular response and sometimes cell degeneration (24). Furthermore, depending on experimental conditions, budding of the virus can occur, which can lead to its spread into adjoining cells and tissues.

Differences in mechanisms of transneuronal migration could also account for the restricted number of neurons labeled by β-gal-TTC. Matteoli et al have provided strong evidence that the intact tetanus toxin crosses the synapses by parasitizing the physiological process of synaptic vesicle recycling at the nerve terminal (22). The toxin probably binds to the inner surface of a synaptic vesicle during the time the lumen is exposed to the external medium. Vesicle endocytosis would then presumably provide the mechanism for internalization of the toxin. Because the TTC fragment is known to mimic the migration of the toxin in vivo, it could therefore direct the fusion protein along a similar transynaptic pathway. If this hypothesis is confirmed, it would strongly suggest that synaptic activity is required for the transneuronal transport of β-gal-TTC. Therefore, only active neuronal circuits would be detected by the hybrid protein. The possible dependence of β-gal-TTC on synaptic vesicle exocytosis and endocytosis could be further investigated, since techniques are now available to record synaptic activity in neural networks in vitro (30). In contrast, the transneuronal pathway of neurotropic viruses has not yet been elucidated and could be fundamentally different, involving virus budding in the vicinity of a synapse. Finally, the transneuronal transport of the hybrid protein might depend on a synaptic specificity, although the tetanus toxin is not known to display any (7, 23). It is therefore likely that a virus would cross different or inactive synapses. In summary, the restricted spectrum of interneuronal transport, in addition to its non-toxicity, make the β-gal-TTC hybrid protein a novel and powerful tool for analysis of neural pathways.

One advantage of the fusion gene of the invention for neuronal mapping is that it derives from a single genetic entity that is amenable to genetic manipulation and engineering. Several years ago, a technique based on homologous recombination in embryonic stem cells was developed to specifically replace genes in the mouse (31, 32). This method generates a null mutation in the substituted gene, although in a slightly modified strategy, a dicistronic messenger RNA can also be produced (33, 34). When a reporter gene, such as E. coli lacZ, is used as the substituting gene, this technique provides a means of marking the mutated cells so that they can be followed during embryogenesis. Thus, this technique greatly simplifies the analysis of both the heterozygote expression of the targeted gene as well as the phenotype of null (homozygous) mutant animals.

Another advantage of this invention is that the composition comprising the fusion gene may encode an antigen or antigens. Thus, the composition may be used to elicit an immune response in its host and subsequently confer protection of the host against the antigen or antigens expressed. These immunization methods are described in Robinson et al., U.S. Pat. No. 5,43,578, which is herein incorporated by reference. In particular, the method of immunizing a patient or animal host comprises introducing a DNA transcription unit encoding comprising the fusion gene of this invention, which encodes a desired antigen or antigens. The uptake of the DNA transcription unit by the host results in the expression of the desired antigen or antigens and the subsequent elicitation of humoral and/or cell-mediated immune responses.

Neural cells establish specific and complex networks of interconnected cells. If a gene were mutated in a given neural cell, we would expect this mutation to have an impact on the functions of other, interconnected neural cells. With these considerations in mind, a genetic marker that can diffuse through active synapses would be very useful in analyzing the effect of the mutation. In heterozygous mutant animals, the cells in which the targeted gene is normally transcribed could be identified, as could the synaptically connected cells of a neural network. In a homozygous animal, the impact of the mutation on the establishment or activity of the neural network could be determined. The feasibility of such an in vivo approach depends critically on the efficiency of synaptic transfer of the fusion protein, as well as its stability and cellular localization.

Another extension of the invention is to gene therapy applied to the CNS. This invention provides for uptake of a non-toxic, enzyme-vector conjugate by axon terminals and conveyance retrogradely to brainstem motoneurons. A selective retrograde transynaptic mechanism subsequently transports the hybrid protein into second-order connected neurons. Such a pathway, which by-passes the blood-brain barrier, can deliver macromolecules to the CNS. In fact, pathogenic agents, such as tetanus toxin and neurotropic viruses, are similarly taken up by nerve endings, internalized, and retrogradely transported to the nerve cell somata. In such a scenario, the lacZ reporter would be replaced by a gene encoding a protein that provides a necessary or interesting activity and/or function. For example, the human CuZn superoxide dismutase, SOD-1, and the human enzyme β-N-acetylhexosaminidase A, HexA, have been fused or chemically coupled to the TTC fragment (13, 16), and their uptake by neurons in vitro was considerably increased and their enzymatic functions partially conserved. Combined with the in vivo experiments described here using β-gal-TTC, a gene therapy approach based on TTC hybrid proteins appears to be a feasible method of delivering a biological function to the CNS. However, ways have to be found to target the TTC hybrid proteins, which are likely to be sequestrated into vesicles, to the appropriate subcellular compartment. Such a therapeutic strategy could be particularly useful for treating neurodegenerative and motoneuron diseases, such as amyotrophy lateral sclerosis (ALS, 35), spinal muscular atrophies (SMA, 36, 37), or neurodegenerative lysosomal storage diseases (38, 39). Injection into selected muscles, even in utero, could help to specifically target the appropriate neurons. In addition, such an approach would avoid the secondary and potentially toxic effects associated with the use of defective viruses to deliver a gene (40, 41).

### Example 1

### Plasmid Constructions

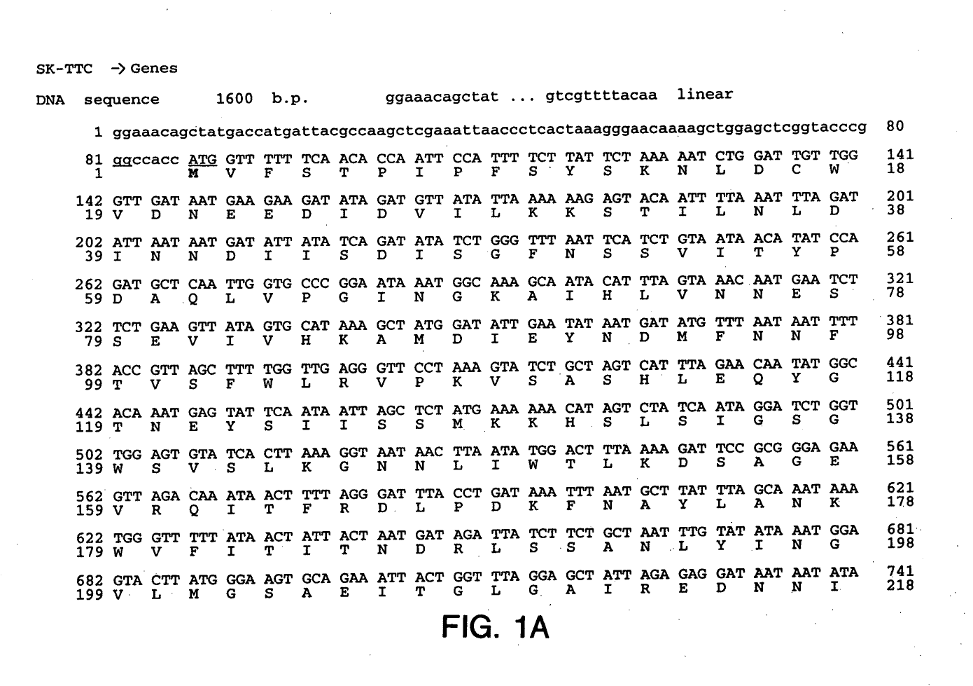

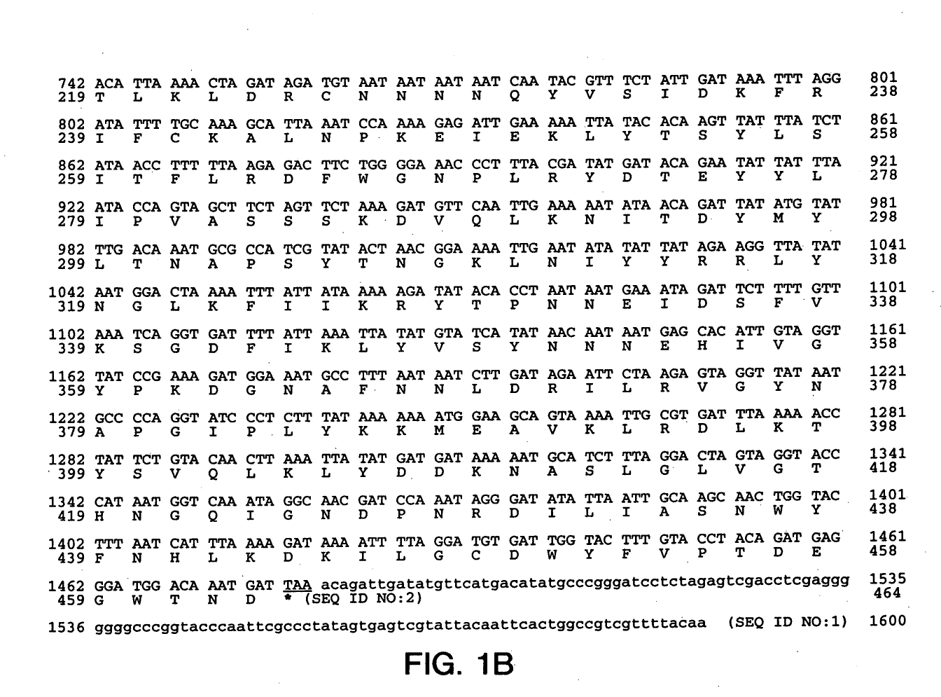

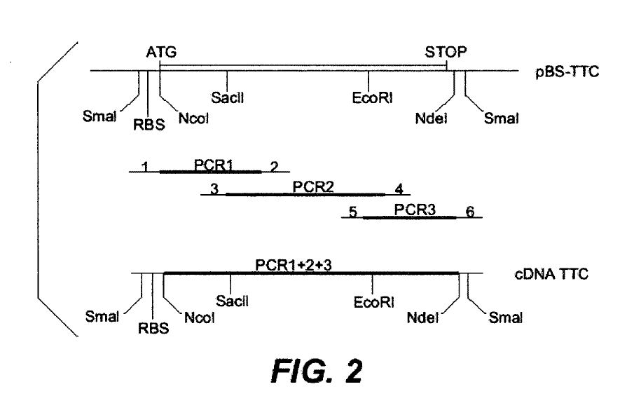

(A) TTC cloning: Full length TTC DNA was generated from the genomic DNA from the Clostridium Tetani strain (a gift from Dr. M. Popoff, Institut Pasteur) using PCR. Three overlapping fragments were synthesized: PCR1 of 465 by (primer 1: 5′-CCC CCC GGG CCA CCA TGG TTT TTT CAA CAC CAA TTC CAT TTT CTT ATT C-3′ and primer 2: 5′-CTA AAC CAG TAA TTT CTG-3′), PCR2 of 648 by (primer 3: 5′-AAT TAT GGA CTT TAA AAG ATT CCG C-3′ and primer 4: 5′-GGC ATT ATA ACC TAC TCT TAG AAT-3′) and PCR3 of 338 by (primer 5: 5′-AAT GCC TTT AAT AAT CTT GAT AGA AAT-3′ and primer 6: 5′-CCC CCC GGG CAT ATG TCA TGA ACA TAT CAA TCT GTT TAA TC-3′). The three fragments were sequentially introduced into pBluescript KS+(Stratagene) to give pBS:TTC plasmid. The upstream primer 1 also contains an optimized eukaryotic Ribosome Binding Site (RBS) and translational initiation signals. Our TTC fragment (462 amino acids) represents the amino acids 854-1315 of tetanus holotoxin, i.e. the carboxy-terminal 451 amino acids of the heavy chain, which constitute the fragment C plus 11 amino acids of the heavy chain that immediately precede the amino terminus of the fragment C. The DNA sequence and amino acid sequence of the TTC fragment cloned in pBS:TTC is shown in FIG. 1. The construct pBS:TTC is shown in FIG. 2.

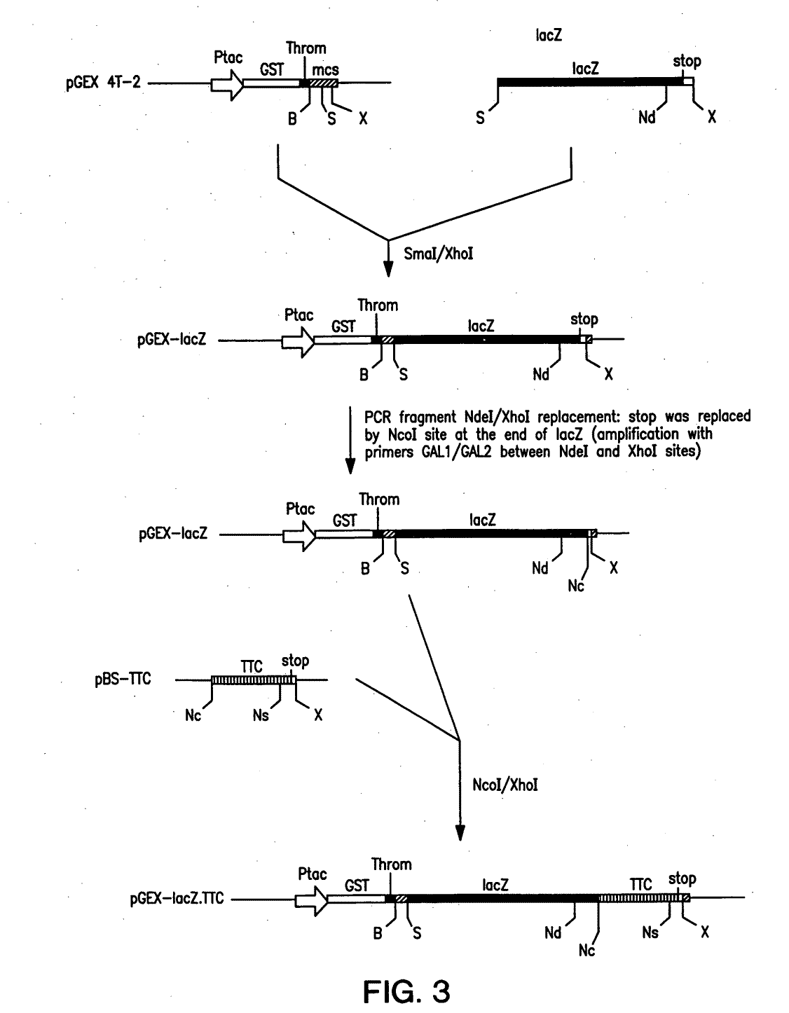

(B) pGEX:lacZ-TTC: pGEX:lacZ was obtained by cloning a SmaI/XhoI lacZ fragment from the pGNA vector (a gift from Dr. H. Le Mouellic) into pGEX 4T-2 (Pharmacia). PCR was used to convert the lacZ stop codon into an NcoI restriction site. Two primers (upstream: 5′-CTG AAT ATC GAC GGT TTC CAT ATG-3′ and downstream: 5′-GGC AGT CTC GAG TCT AGA CCA TGG CTT TTT GAC ACC AGA C-3′) were used to amplify the sequence between NdeI and XhoI, generating pGEX:lacZ(NcoI) from pGEX:lacZ pGEX:lacZ-TTC was obtained by insertion of the TTC NcoI/XhoI fragment into pGEX:lacZ(NcoI), fusing TTC immediately downstream of the lacZ coding region and in the same reading frame. FIG. 3 shows the details of the pGEX:lacZ-TTC construct.

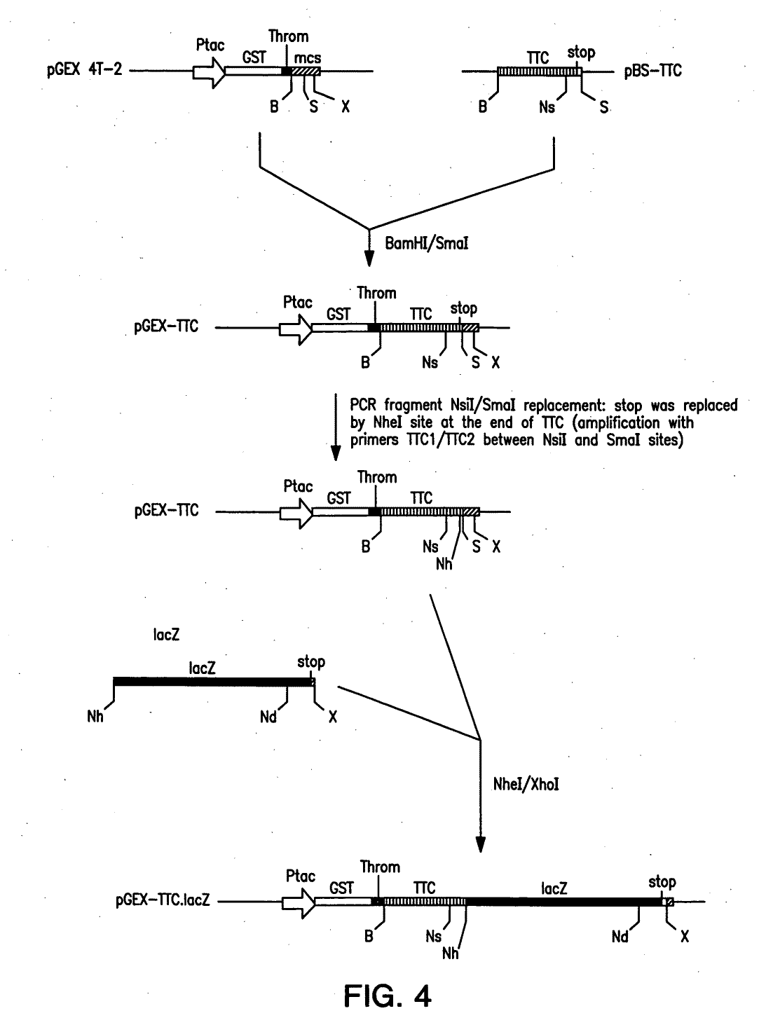

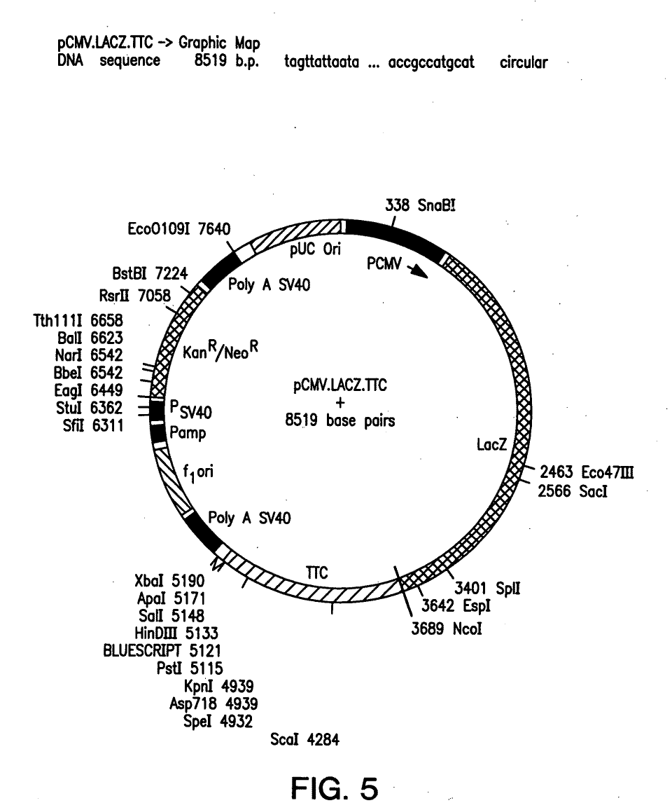

(C) pGEX:TTC-lacZ: pBS:TTC was modified to change NcoI into a BamHI restriction site (linker 5′-CAT GAC TGG GGA TCC CCA GT-3′) at the start of the TTC DNA, to give pBS:TTC(BamHI) plasmid. pGEX:TTC was obtained by cloning The TTC BamHI/SmaI fragment from pBS:TTC(BamHI) into pGEX 4T-2 (Pharmacia). PCR was used to convert the TTC stop codon into an NheI restriction site. Two primers (upstream: 5′-TAT GAT AAA AAT GCA TCT TTA GGA-3′ and downstream: 5′-TGG AGT CGA CGC TAG CAG GAT CAT TTG TCC ATC CTT C-3′) were used to amplify the sequence between NsiI and SmaI, generating pGEX:TTC(NheI) from pGEX:TTC. The lacZ cDNA from plasmid pGNA was modified in its 5′ extremity to change SacII into an NheI restriction site (linker 5′-GCT AGC GC-3′). pGEX:TTC-/acZwas obtained by insertion of the lacZ NheI/XhoI fragment into pGEX:TTC(NheI), fusing lacZ immediately downstream of the TTC coding region and in the same reading frame. The details of the construct of pGEX:TTC-/acZ are shown in FIG. 4.

(D) pCMV:lacZ-TTC: pCMV vector was obtained from pGFP-C1 (Clontech laboratories) after some modifications: GFP sequence was deleted by a BgIII/NheI digestion and relegation, and SacII in the polylinker was converted into an AscI restriction site (linkers 5′-GAT ATC GGC GCG CCA GC-3′ and 5′-TGG CGC GCC GAT ATC GC-3′).

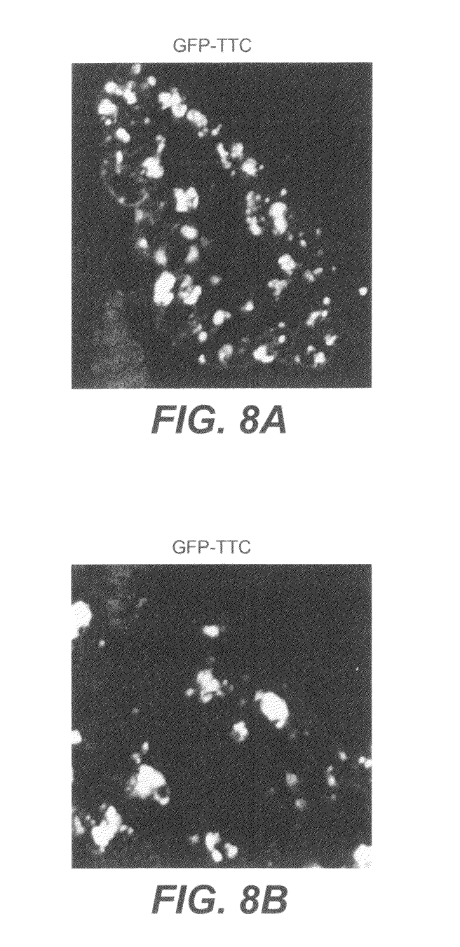

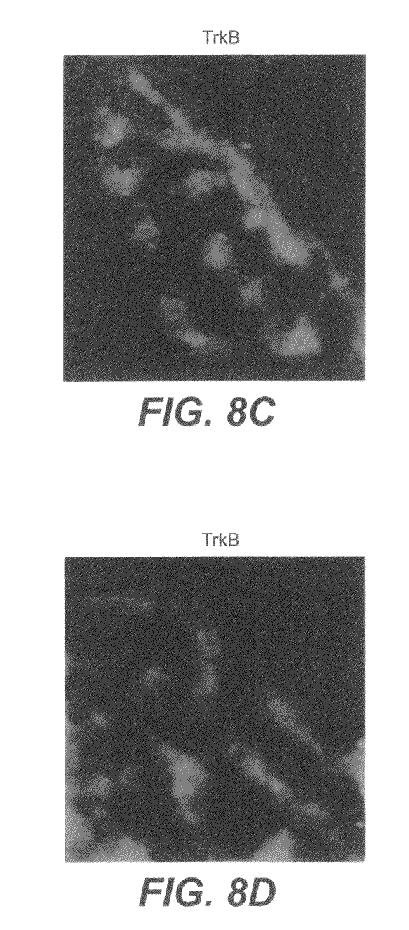

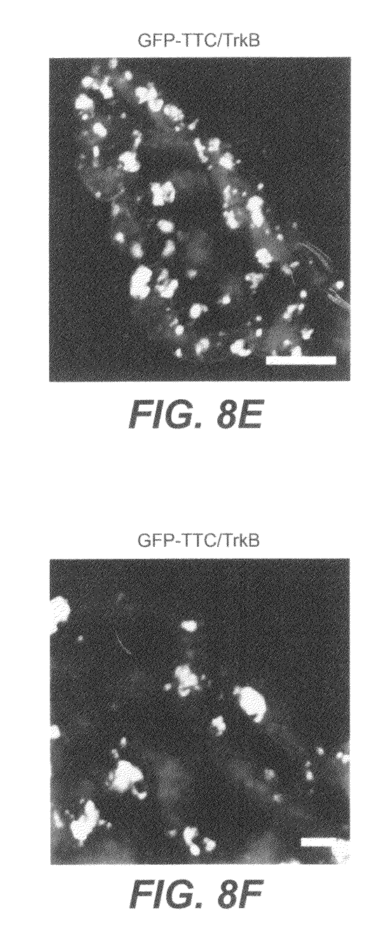

pBluescript KS+(Stratagene) was modified to change XhoI into an AscI restriction site (linker 5′-TCG ATG GCG CGC CA-3′), giving pBS(AscI) plasmid. pBS:lacZ-TTC was obtained by cloning a XmaI lacZ-TTC fragment from pGEX:lacZ-TTC into pBS(AscI). pCMV:lacZ-TTC was obtained by insertion of the lacZ-TTC XmnI/AscI fragment into pCMV vector at the XhoI and AscI sites (XhoI and XmnI was eliminated with the clonage), putting the fusion downstream of the CMV promoter. FIG. 8 shows the details of the construct pCMV:lacZ-TTC. Plasmid pCMV:lacZ-TTC was deposited on Aug. 12, 1997, at the Collection Nationale de Cultures de Microorganisms (CNCM), Institut Pasteur, 25, Rue de Docteur Roux, F-75724, Paris Cedex 15, France, under Accession No. I-1912.

### Example 2

### Purification of the Hybrid Protein

The E. coli strain SR3315 (a gift from Dr. A. Pugsley, Institut Pasteur) transfected with pGEX:lacz-TTC was used for protein production. An overnight bacterial culture was diluted 1:100 in LB medium containing 100 μg/ml ampicillin, and grown for several hours at 32° C. until an OD of 0.5 was reached. Induction from the Ptac promoter was achieved by the addition of 1 mM IPTG and 1 mM MgCl₂ and a further 2 hrs incubation. The induced bacteria were pelleted by centrifugation for 20 min at 3000 rpm, washed with PBS and resuspended in lysis buffer containing 0.1 M Tris pH 7.8, 0.1 M NaCl, 20% glycerol, 10 mM EDTA, 0.1% Triton-X100, 4 mM DTT, 1 mg/ml lysozyme, and a mixture of anti-proteases (100 μg/ml Pefablok, 1 μg/ml leupeptin, 1 μg/ml pepstatin, 1 mM benzamidine). After cell disruption in a French Press, total bacterial lysate was centrifuged for 10 min at 30000 rpm. The resulting supernatant was incubated overnight at 4° C. with the affinity matrix Glutathione Sepharose 4B (Stratagene) with slow agitation. After centrifugation for 5 min at 3000 rpm, the matrix was washed three times with the same lysis buffer but without lysozyme and glycerol, and then three times with PBS. The resin was incubated overnight at 4° C. with Thrombin (10 U/ml; Sigma) in PBS in order to cleave the β-gal-TTC fusion protein from the Glutatione-S-transferase (GST) sequence and thereby elute it from the affinity column. Concentration of the eluted fusion protein was achieved by centrifugation in centricon X-100 tubes (Amicon; 100,000 MW cutoff membrane).

Purified hybrid protein was analyzed by Western blotting after electrophoretic separation in 8% acrylamide SDS/PAGE under reducing conditions followed by electrophoretic transfer onto nitrocellulose membranes (0.2 mm porosity, BioRad). Immunodetection of blotted proteins was performed with a Vectastaln ABC-alkaline phosphatase kit (Vector Laboratories) and DAB color development. Antibodies were used as follows: rabbit anti-β-gal antisera (Capel), dilution 1:1000; rabbit anti-TTC antisera (Calbiochem), dilution 1:20000. A major band with a relative molecular mass of 180 kDa corresponding to the β-Gal-TIC hybrid protein was detected with both anti* Gal anti-TTC antibodies.

### Example 3

### Binding and Internalization of Recombinant Protein in Differentiated 1009 Cells

The 1009 cell line was derived from a spontaneous testicular teratocarcinoma arising in a recombinant inbred mouse strain (129×B6) (17). The 1009 cells were grown in Dulbecco's modified Eagle's medium (DMEM) containing 10% fetal calf serum and passaged at subconfluence. In vitro differentiation with retinoic acid and cAMP was performed as described (18). Eight days after retinoic acid treatment, cells were used for the internalization experiments with either the hybrid protein or β-gal.

Binding and internalization of the β-Gal-TIC fusion were assessed using a modified protocol (16). Differentiated 1009 cells were incubated for 2 hrs at 37° C. with 5 μg/ml of 13-Gal-TTC or 13-Gal protein diluted in binding buffer (0.25% sucrose, 20 mM Tris acetate 1 mM CaCl₂, 1 mM MgCl₂, 0.25% bovine serum albumin, in PBS). The cells were then incubated with 1 μg/ml Pronase E (Sigma) in PBS for 10 min at 37° C., followed by washing with proteases inhibitors diluted in PBS (100 μg/ml Pefablok, 1 mM benzamidine).

The cells were fixed with 4% formalin in PBS for 10 min at room temperature (RT) and then washed extensively with PBS. β-gal activity was detected on fixed cells by an overnight staining at 37° C. in X-Gal solution (0.8 mg/ml X-Gal, 4 mM potassium ferricyanide, 4 mM potassium ferrocyanide, 4 mM MgCl₂ in PBS). For electron microscopy, the cells were further fixed in 2.5% glutaraldehyde for 18 hrs, and then processed as described (19).

For immunohistochemical labeling, cells were fixed with 4% paraformaldehyde in PBS for 10 min at RT then washed extensively with PBS, followed by a 1 hr incubation at RT with 2% BSA/0.02% Triton X-100 in PBS. Cells were co-incubated in primary antibodies diluted in 2% BSA/0.02% Triton X-100 in PBS for 2 hrs at RT. Antibodies used were a mouse anti-neurofilament antibody (NF 200 K_{d}, dilution 1:50; Sigma) or the rabbit anti-TTC antibody (dilution 1:1000). The labeling was visualized using fluorescent secondary antibodies: Cy3, goat anti-rabbit IgG (dilution 1:500; Amersham) or anti-mouse IgG with extravidin-FITC (dilution 1:200; Sigma). Cells were mounted in moviol and visualized with epifluorescence.

### Example 4

### In Vivo Recombinant Protein Injection

14-week old B6D2F1 mice were obtained from IFFA-CREDO. The animal's tongue muscle was injected using an Hamilton syringe (20 μl per animal) while under general anesthesia with 3% Avertin (15 μl/g of animal). The protein concentration was 0.5 to 5 μg/μl in PBS; therefore, mice received approximately 10 to 100 μg per injection. Animals were kept alive for 12 hrs to 48 hrs post-injection to permit migration of the injected protein, and in no case were any tetanus symptoms detected. The mice were sacrificed by intracardiac perfusion with 4% paraformaldehyde in PBS while under deep anesthesia. Brains were harvested, rinsed in PBS and incubated in 15% sucrose overnight at 4° C., then mounted in tissue-tek before sectioning, 15 μm thick slices using a cryostat.

### Example 5

### Histology, Immunohistology, and X-Gal Staining

For in toto X-Gal staining of the dissected brain and tongue, mice (10 animals) were sacrificed and fixed as described above. The brain was further cut with a scalpel along a median plane and directly incubated for 12 hrs in X-Gal solution.

For immunohistology, sections were incubated In a 1:5000 dilution of anti-TTC antibody in 2% BSA/0.02% Triton X-100 in PBS overnight at 4° C. after nonspecific antibody binding sites were blocked by a 1 hr incubation in the same buffer. Antibody detection was carried out using the Vectastain ABC-alkaline phosphatase kit with DAB color development. For X-Gal staining, sections were incubated in X-Gal solution and counterstained for 30 sec with hematoxylin 115 (v/v) in PBS. Histology on adjacent sections was done after X-Gal staining, using a 30 sec incubation in hematoxylin/thionin solution. All sections were mounted in moviol before eight microscopy analysis.

### Example 6A

### Internalization of the β-Gal-TTC Fusion Protein by Neurons In Vitro

Differentiation of 1009 cells with retinoic acid and cAMP in vitro yields neuronal and glial cells (18, 20). X-Gal staining or immunolabeling were performed after incubation with the β-gal-TTC fusion protein or with either the β-gal or TTC proteins alone. Only when the hybrid protein was incubated with differentiated 1009 cells was a strong X-Gal staining detected in cells having a neuronal phenotype. No signal was detected when β-gal alone was incubated under the same conditions. A similar X-Gal staining pattern was obtained after pronase treatment of the cells to remove surface bound proteins, indicating that the hybrid protein had been internalized. The intracellular localization of the hybrid protein was further confirmed by electron microscopic analysis of X-Gal-stained cells. Furthermore, the enzymatic activity observed in axons seemed to be localized in vesicles associated with filaments, which is in agreement with previous work on TIC fragment or native tetanus toxin (14, 21, 22). Co-labeling with anti-TIC and anti-neurofilament antibodies revealed that β-gal activity co-localized with TTC fragment in neuronal cells. No glial cells were labeled with either antibody.

### Example 6B

### Internalization of the TTC-β-gal Fusion Protein by Neurons In Vitro

The method used for the internalization was identical to that described in Example 6 above. The results show efficiently internalization of the hybrid as in Example 6 above.

### Example 7

### Retrograde Transport of the Hybrid Protein In Vivo

To study the behavior of the β-gal-TTC protein in vivo, the hybrid protein was tested in a well characterized neuronal network, the hypoglossal system. After intramuscular injection of β-gal-TTC protein into the mouse tongue, the distribution of the hybrid protein in the CNS was analyzed by X-Gal staining. Various dilutions of the protein were injected and sequential time points were analyzed to permit protein transport into hypoglossal motoneurons (XII), and its further transneuronal migration into connected second order neurons.

A well-defined profile of large, apparently retrogradely labeled neurons was clearly evident in the hypoglossal structure, analyzed in toto at 12 hrs post-injection. A strong labeling was also apparent in the hypoglossal nerve (XIIn) of the tongue of the injected mice. At the level of muscle fibers, button structures were observed that might reflect labeling of neuromuscular junctions where the hybrid protein was internalized into nerve axons. These data demonstrate that the β-gal-TTC hybrid protein can migrate rapidly by retrograde axonal transport as far as motoneuron cell bodies, after prior uptake by nerve terminals in the tongue. This specific uptake and the intra-axonal transport are similar to the properties that have been described for the native toxin (6, 21, 23).

Transport of the hybrid protein was examined in greater detail by analyzing X-Gal-stained brain sections. Motoneurons of the hypoglossal nucleus became labeled rapidly, with 12 hrs being the earliest time point examined. Most of the label was confined to neuronal somata, the cell nuclei being unlabeled. The intensity of the labeling depends upon the concentration of the β-gal-TTC protein injected: when 10 μg of protein was injected, only the hypoglossal somata were detected, whereas with 25 to 50 μg a fuzzy network of dendrites was visualized; transynaptic transfer was detected with 100 μg of hybrid protein. An identical distribution of label was observed then brain sections were immunostained with an anti-TTC antibody, demonstrating that β-gal and TTC fragment co-localize within cells. Finally, injection of β-gal alone did not result in labeling of the hypoglossal nuclei and therefore confirms that transport of the hybrid protein is TTC-dependent. Labeling with an anti-TTC antibody was less informative than detection of β-gal activity; for instance, the nerve pathway to the brain could not be visualized by anti-TTC immunostaining. At 18 hrs post-injection, labeling was observed in the hypoglossal nuclei: all motoneuron cell bodies and the most proximal part of their dendrites were very densely stained. In contrast, no labeling was ever detected in glial cells adjoining XII motoneurons or their axons. Our results are in accordance with others who reported an identical pattern of immunolabeling after injection of the TTC fragment alone (9). Transneuronal transfer is detectable after 24 hrs. An additional 24 hrs and beyond did not yield a different staining.

### Example 8

### Transneuronal Transport of the Hybrid Protein

Second order interneurons, as well as higher order neurons that synapse with the hypoglossal motoneurons, have been extensively analyzed using conventional markers, such as the wheat germ agglutinin-horseradish peroxidase complex (WGA-HRP) or neurotropic viruses such as alpha-herpes (24) and rhabdoviruses (25). An exhaustive compilation of regions in the brain that synaptically connect to the hypoglossal nucleus has also been described recently (25). In this invention, the distribution of the β-gal-TTC fusion depended on the initial concentration of protein injected into the muscle and the time allowed for transport after injection. Up to 24 hrs post-injection, labeling was restricted to the hypoglossal nuclei. After 24 hrs, the distribution of second order transneuronally labeled cells in various regions of the brain was consistent and reproducible. Even at longer time points (e.g. 48 hrs), labeling of the hypoglossal nucleus remained constant. At higher magnification, a discrete and localized staining of second-order neurons was observed, suggesting that the hybrid protein had been targeted to vesicles within cell somata, synapses and axons. A similar patchy distribution was previously described for tetanus toxin and TTC fragment alone (14, 21, 22).

Intense transneuronal labeling was detected in the lateral reticular formation (LRF), where medullary reticular neurons have been reported to form numerous projections onto the hypoglossal nucleus (26, 27). β-gal activity was detected bilaterally in these sections. Label led LRF projections formed a continuous column along the rostrocaudal axis, beginning lateral to the hypoglossal nucleus, with a few neurons being preferentially stained in the medullary reticular dorsal (MdD) and the medullary reticular ventral (MdV) nuclei. This column extends rostrally through the medulla, with neurons more intensely labeled in the parvicellular reticular nucleus (PCRt, caudal and rostral). After 48 hrs, cells in MdD and PCRt were more intensely stained. A second bilateral distribution of medullary neurons projecting to the hypoglossal nucleus was detected in the solitary nucleus (SoI) but the labeling was less intense than in the reticular formation, presumably because relatively few cells of the solitary nucleus project onto the hypoglossal nucleus (26). However, no labeling was found in the spinal trigeminal nucleus (Sp5), which has also been shown to project onto the hypoglossal nucleus (26). Transynaptic transport of the β-gal-TTC protein was also detected in the pontine reticular nucleus caudal (PnC), the locus coeruleus (LC), the medial vestibular nucleus (MVe) and in a few cells of the inferior vestibular nucleus (IV). These cell groups are known to project onto the hypoglossal nucleus (25), but their labeling was weak, probably because of the greater length of their axons. A few labeled cells were observed in the dorsal paragigantocellular nucleus (DPGi), the magnocellular nucleus caudal (RMc), and the caudal raphe nucleus (R); their connections to the hypoglossal nucleus have also been reported (25). Finally, labeled neurons were detected bilaterally in midbrain projections, such as those of the mesencephalic trigeminal nucleus (Me5), and a few neurons were stained in the mesencephalic central gray region (CG). These latter nuclei have been typed as putative third order cell groups related to the hypoglossal nucleus (25).

Neurons in the motor trigeminal nucleus (Mo5) and the accessory trigeminal tract (Acs5) were also labeled, along with a population of neurons in the facial nucleus (N7). However, interpretation of this labeling is more ambiguous, since it is known that motoneurons in these nuclei also innervate other parts of the muscular tissue, and diffusion of the hybrid protein might have occurred at the point of injection. Conversely, these nuclei may have also projected to the tongue musculature via nerve XII, since neurons in N7 have been reported to receive direct hypoglossal nerve input (28). This latter explanation is consistent with the fact that labeling in these nuclei was detected only after 24 hrs; however, this point was not further investigated.

Together, the data summarized in Table 1 clearly establish transneuronal transport of the β-gal-TTC fusion protein from the hypoglossal neurons into several connected regions of the brainstem.

TABLE 1
Transneuronal transport of the lacZ-TTC fusion from the XII nerve:
labeling of different cells types in the central nervous system.

| | | |
|---|---|---|
| Cell groups | 12-18 hrs | 24-48 hrs |

First order neurons
First category:

| | | |
|---|---|---|
| XII, hypoglossal motoneurons | ++ | +++ |

Second category:

| | | |
|---|---|---|
| N7, facial nu | − | ++ |
| Mo5, motor trigeminal nu | − | ++ |
| Acs5, accessory trigeminal nu | − | + |

Second order cell groups

| | | |
|---|---|---|
| MdD, medullary reticular nu, dorsal | − | ++ |
| MdV, medullary reticular nu, ventral | − | +/− |
| PCRt, parvicellular reticular nu, caudal | − | ++ |
| PCRt, parvicellular reticular nu, rostral | − | ++ |
| Sol, solitary tract nu | − | + |
| DPGi, dorsal paragigantocellular nu | − | +/− |
| PnC, pontine reticular nu, caudal | − | + |
| RMc, magnocellular reticular nu | − | +/− |
| R, caudal raphe nu | − | +/− |
| MVe, medial vestibular nu | − | + |
| IV, inferior vestibular nu | − | +/− |
| LC, locus coeruleus | − | + |
| Me5, mesencephalic trigeminal nu (*) | − | + |
| CG, mesenphalic central gray (*) | − | +/− |

(*) Represents second order cell groups that also contain putative third order neurons (see text).
−, no labeling;
+ to +++, increased density of label;
+/− weak labeling. 16 animals were analyzed for the 12-18 hrs p.i. data;
6 animals were analyzed for the 24-48 hrs p.i. data.

In another embodiment of the invention, we have constructed a fusion protein (GFP-TTC) comprising the C-terminal fragment of tetanus toxin and the GFP reporter gene, and have demonstrated its effectiveness to map a simple neural network retrogradely and transsynaptically in transgenic mice. (Maskos et al., 2002). The GFP-TTC fusion protein permits the visualization of membrane traffic at the presynaptic level of the neuromuscular junction and can be detected optically without immunological or enzymatic reactions. The GFP-TTC fusion protein, therefore, permits observation of active neurons with minimal disturbance of their physiological activities.

We have also previously shown that, without neural activity, localization of a TTC fusion protein at the NMJ is impaired (Miana-Mena et al., 2002). In this aspect of the invention, therefore, we investigated in vivo, the influence of neurotrophic factors on neuronal localization and internalization of GFP-TTC and the mechanisms by which certain neurotrophic factors influence neuronal trafficking in vivo. We found that localization of GFP-TTC at the NMJ is rapidly induced by neurotrophic factors such as Brain Derived Neurotrophic Factor (BDNF), Neurotrophin 4 (NT-4), and Glial-Derived Neurotrophic Factor (GDNF) but not by Nerve Growth Factor (NGF), Neurotrophin 3 (NT-3), and Ciliary Neurotrophic Factor (CNTF).

Co-injection of various amounts of BDNF with the GFP-TTC probe induces an increase of the fluorescence measured at the neuromuscular junction (NMJ). This effect, which is detectable as early as 5 min after injection and reaches a maximum level at about 30 min after injection, indicates that BDNF treatment enhances neuronal endocytosis. Among other functions, BDNF stimulates the secretion of neurotransmitter from Xenopus nerve muscle co-cultures and from hippocampal neurons (Lohof et al., 1993; Tyler and Pozzo-Miller, 2001). Since tetanus toxin is known to enter neurons by means of synaptic vesicle endocytosis (Matteoli et al., 1996), BDNF might increase GFP-TTC internalization through enhancement of synaptic vesicle turnover. In our study, BDNF effects persisted after BoTx/A treatment, which blocks exocytosis and endocytosis of synaptic vesicles, showing that BDNF increases the kinetics and localization of a TTC-containing fusion protein at the NMJ through another endocytic pathway. Therefore, intramuscular injection of GFP-TTC and visualization of transport mechanisms revealed at least two different endocytic pathways: a clathrin-dependent and a clathrin-independent pathways. We found that after intramuscular injection of GFP-TTC, it displayed characteristics consistent with localization in lipid rafts, including biochemical co-localization with caveolin 3 and co-localization with GM1, a raft marker identified by CT-b binding (Orlandi and Fishman, 1998; Wolf et al., 1998). Accordingly, the clathrin-independent pathway used by GFP-TTC, appears to involve lipid microdomains. Analysis by confocal microscopy revealed morphologically two different labelings. Firstly, a GFP-TTC diffuse staining, which partially overlaps with the synaptic vesicle SV2 but also with the raft marker CT-b, indicating a mixing of synaptic vesicles and lipid rafts. Secondly, highly fluorescent domains, which are detected before and persist after the more diffuse pattern and that appear to be invaginations or infoldings of the synaptic membrane. These GFP-TTC patches contained only CT-b labeling. Indeed, lipid microdomains which play a role in cellular functions such as vesicular trafficking and signal transduction (Simons and Toomre, 2000), can move laterally and cluster into larger patches (Harder et al., 1998). They might also be specific zones of exocytosis in the presynaptic compartment, undergoing a rapid form of internal traffic in response to retrograde signaling from target cells. Similar infolding and cisternae structures have been described in frog motor nerve terminals which replenish the pool of synaptic vesicles in a manner dependent upon neuronal activity (Richards et al., 2000). In CHO cells, tubular caveolae have also been described (Mundy et al., 2002).

Based on the kinetics of probes for NMJ localization, we observed different trafficking behaviors for GFP-TTC and CT-b. It has been postulated that targeting of toxin into the cell depends on the structure and function of endogenous ganglioside receptors, which could couple toxins to specific lipid raft microdomains (Wolf et al., 1998). Thus, in vivo, endogenous or injected BDNF might increase the amount of lipid microdomains containing TTC receptors. Tetanus toxin and cholera toxin bind to different gangliosides, known as GD1b/GT1b and GM1, respectively. Hence, the difference we observed in the dynamics of recruitment at the presynaptic motor nerve terminal may be relevant to different lipid microdomains having specific glycosphingolipids and protein composition. Neuronal membranes are rich in gangliosides and different microdomains are likely to co-exist on the cell surface. Indeed, Thy-1 and PrP prion protein, two functionally different GPI proteins, are found in adjacent microdomains (Madore et al., 1999). Similarly, syntaxins are concentrated in cholesterol-dependent microdomains, which are distinct from rafts containing GPI-linked proteins (Lang et al., 2001).

Like BDNF, NT-4 was also found to increase the concentration of GFP-TTC at the NMJ, whereas NGF and NT-3 had no effect. Since the TrkB receptor is specifically activated by BDNF and NT-4, TrkB activation might be involved in this neoronal trafficking. Interestingly, high-frequency neuronal activity and synaptic transmission have been shown to elevate the number of TrkB receptors on the surface of cultured hippocampal neurons (Du et al., 2000), apparently by recruiting extra TrkB receptors to the plasma membrane (Meyer-Franke et al., 1998). Moreover, TrkB is highly enriched in lipid microdomains from neuronal plasma membrane (Wu et al., 1997). However, no specific co-localization between GFP-TTC and TrkB or p-Trk receptors were detected at the NMJ. Thus, TrkB may act indirectly on the detected traffic at the presynaptic motor nerve membrane.

It is worth noting that the TTC fragment has been detected in cultured motoneurons in the same vesicles as p75^{NTR} (Lalli and Schiavo, 2002). This co-localization may be explained by the tight association of p75^{NTR}, which is expressed mainly during development and in pathological conditions, with GT1b ganglioside (Yamashita et al., 2002). Binding of neurotrophins to their Trk receptors leads to phosphorylation of tyrosine residues that are recognized by several intracellular signaling proteins. Such interactions lead to the activation, by means of a kinase cascade, of the MAP kinase, PI 3-kinase and phospholipase-C-γ pathways (for review see (Huang and Reichardt, 2003)). Many of the intermediates in these signaling cascades are also present in lipid rafts (Simons and Toomre, 2000; Tsui-Pierchala et al., 2002). Activation of PKA is required for translocation of activated p75^{NTR} to lipid rafts (Higuchi et al., 2003). Similarly, the coreceptor GFRα1, which binds GDNF and thus allows activation of the c-RET tyrosine kinase receptor, localize to lipid rafts. GFRα1 recruits RET to lipid rafts after GDNF stimulation and results in strong and continuous signal transduction (Paratcha et al., 2001; Tansey et al., 2000).

Another neurotrophic factor, GDNF, also induced GFP-TTC localization at the NMJ. GDNF, however, activates a different receptor (i.e., a GFRα/cRET receptor) than BDNF and NT-4. Because BDNF/NT-4 and GDNF activate different receptors, we postulated that component(s) of their activation pathways may activate the trafficking of GFP-TTC receptors in specific lipid microdomains. Indeed, various stimuli can lead to internalization of caveolae, a specialized form of lipid rafts. Thus, simian virus 40 stimulates its internalization in caveolae and transport via caveosomes (Pelkmans et al., 2001). Similarly, the albumin-docking protein μg60 activates its transendothelial transport by interaction with caveolin-1 and subsequent activation of Src kinase signaling (Minshall et al., 2000). Recently, it has been reported that tetanus toxin can activate, through the TTC fragment, intracellular pathways involving Trk receptors, extracellular signal-regulated kinases (ERK) and protein kinase C isoforms (Gil et al., 2001; Gil et al., 2000; Gil et al., 2003). In this way, tetanus toxin could therefore autoactivate its neuronal endocytosis via an uncoated pathway rather than by clathrin-dependent pathway to avoid the lysosomal degradation.

Finally, we have demonstrated that GFP-TTC trafficking is regulated by neurotrophic factors. By visualization of GFP-TTC trafficking, our data show that BDNF can stimulate both clathrin-coated and uncoated endocytic pathways, presumably via TrkB activation. Since tetanus toxin, as other pathogens or toxins, uses constitutive mechanisms for its internalization and traffic in cells, we have been able to visualize with GFP-TTC, a physiological response to neurotrophic factors.

This aspect of the invention is further discussed in the following examples.

### Example 9

### GFP-TTC Localization at the NMJ

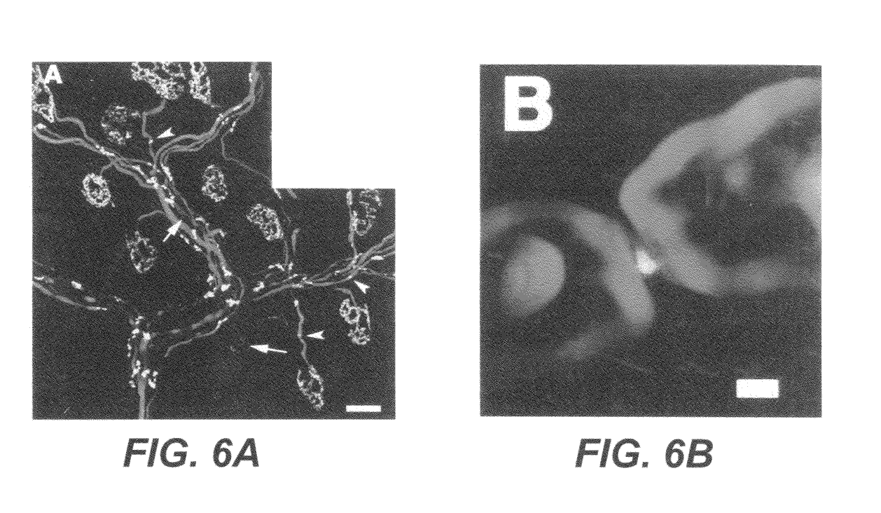

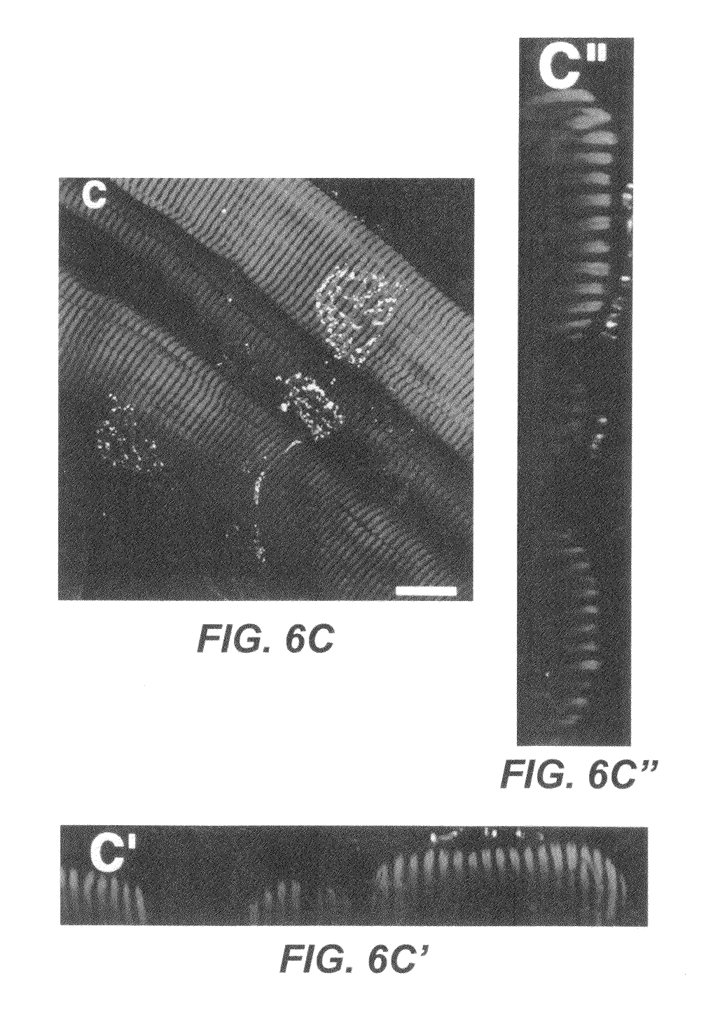

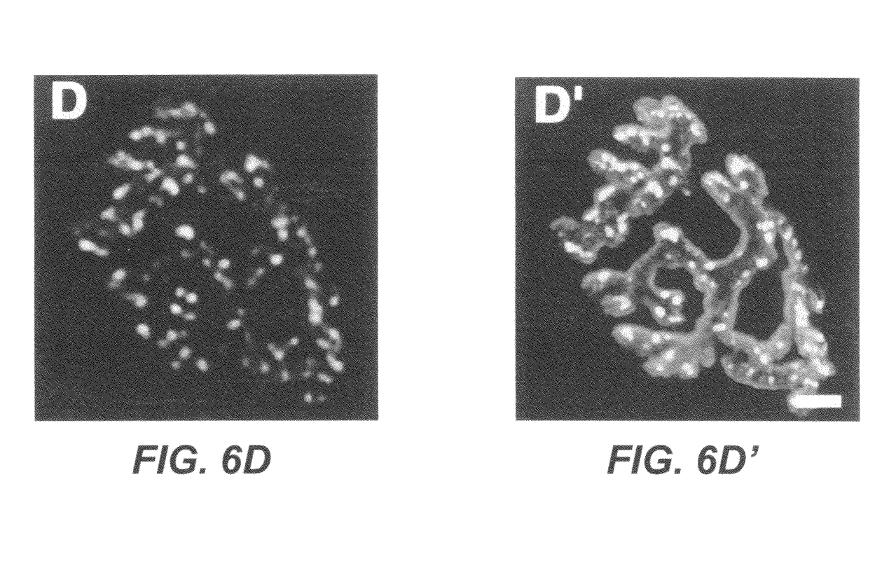

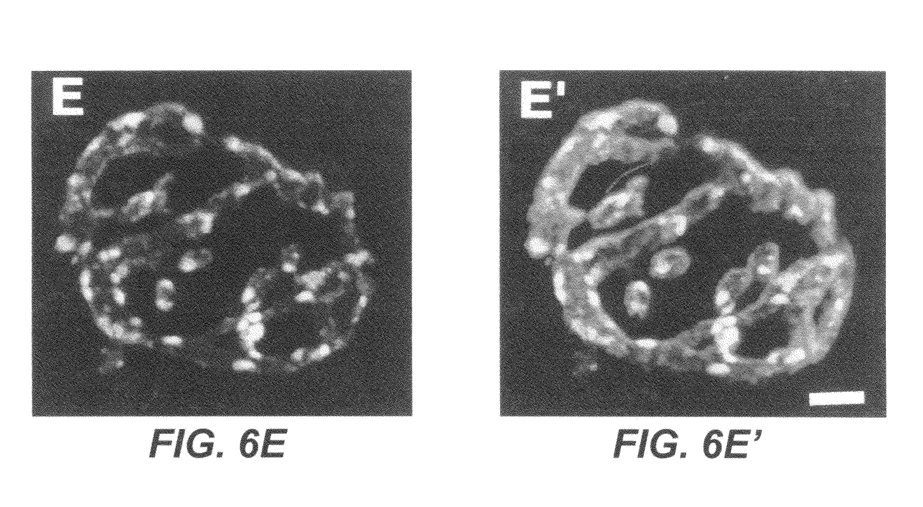

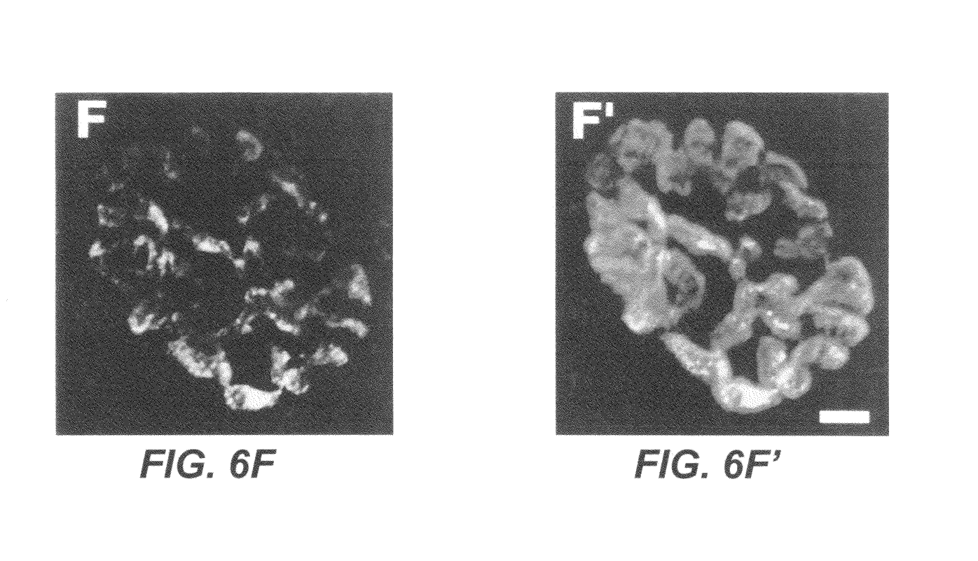

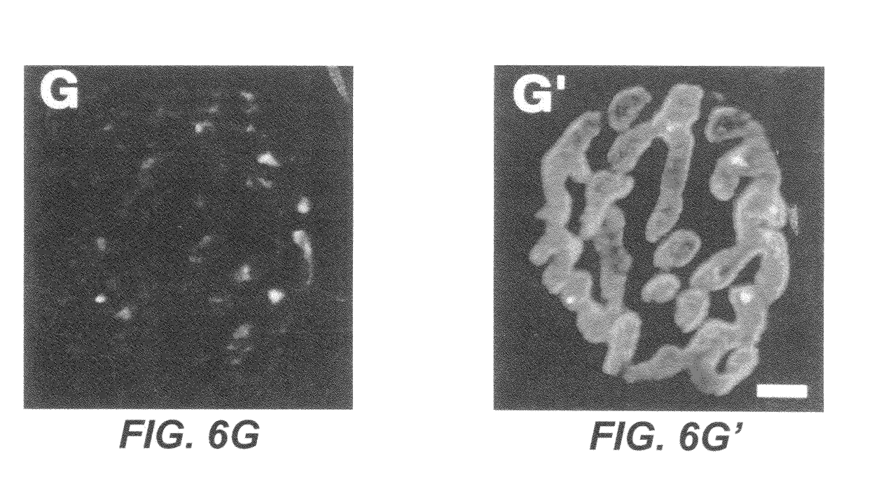

To determine the characteristics of the GFP-TTC distribution at the NMJ, a single injection of the GFP-TTC fusion protein was performed in the immediate vicinity of the Levator auris longus (LAL) muscle and at various times after the injection, the LAL was removed and examined as a whole mount. As LAL is a thin and flat muscle consisting of only a few layers of fibers, the entirety of the neuromuscular preparation with associated nerves could be examined by confocal analysis (FIG. 6A). As shown in FIG. 6, GFP-TTC rapidly concentrates at the NMJ, as identified by the staining of muscle nicotinic acetylcholine receptors with TRITC-conjugated α-bungarotoxine (α-BTX). A patchy clustering of GFP-TTC was observed after approximately 5 min following the deposit of the fusion protein onto the surface of the LAL muscle (FIGS. 6D and D′). After 30 min, a more diffuse staining was observed that was distributed over the entire surface of the NMJ (FIGS. 6E and E′), and which persisted for about 2 h (FIGS. 6F and F′). Immunostaining experiments, performed with an antibody that recognizes troponin T confirmed that GFP-TTC is concentrated mainly in presynaptic motor nerve terminals of the NMJ (FIGS. 6C and C′). We could also detect a strong GFP-TTC labeling at the nodes of Ranvier of intramuscular myelinated axons and in sensory nerve fibers (FIG. 6A and B; arrows and arrowheads respectively). It is likely that most of the GFP-TTC probe was internalized within 24 h, since only a few fluorescent patches persisted at the NMJ 24 h after its injection (FIGS. 6G and G′).

### Example 10

### Influence of BDNF on GFP-TTC Trafficking in Motor Nerve Terminals

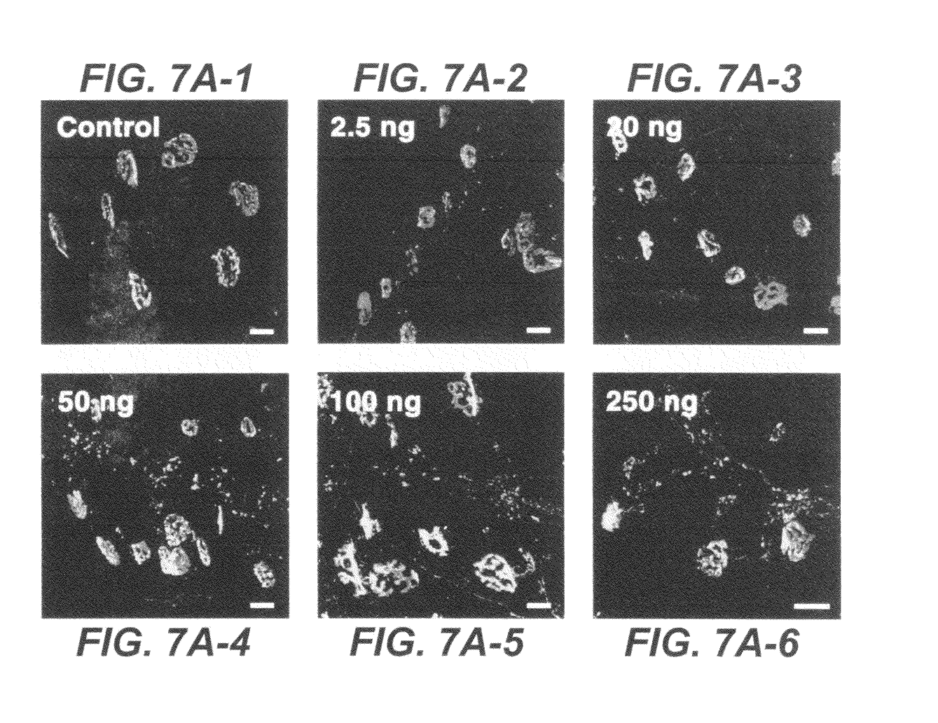

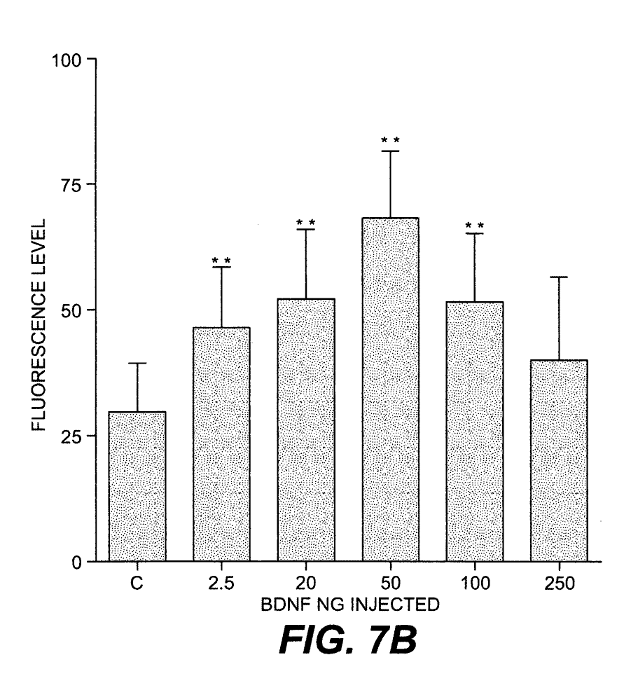

To assess whether exogenously applied neurotrophins affected GFP-TTC recruitment in motor nerve terminals, increasing concentrations of BDNF (2.5-250 ng) were co-injected with GFP-TTC in the vicinity of LAL muscles, while control mice were injected with GFP-TTC alone. Mice were sacrificed and LAL muscles harvested 30 min after injection. GFP fluorescence was quantified by confocal microscopy analysis at NMJs, after identification by TRITC-α-BTX labeling. BDNF injection produced a statistically significant concentration-dependent enhancement of GFP-TTC fluorescence at the NMJ, with the highest effect obtained with 50 ng BDNF (FIG. 7B and Table 2) while higher doses (100 and 250 ng) resulted in weaker elevations in the level of GFP-TTC concentration at the NMJ (1.72±0.12 and 1.15±0.22 fold respectively). The higher GFP-TTC axonal labeling observed at these higher doses (FIG. 7A, arrows), probably correlates to an enhanced internalization of the probe.

In TrkB mutant mice, a physiological phenotype in the facial nerve nucleus, which innervates LAL muscle has been reported (Klein et al., 1993; Silas-Santiago et al., 1997). To exclude the possibility that the BDNF effect observed could be LAL specific, a different muscle, the gastrocnemius, was also analyzed. Thirty minutes after injecting GFP-TTC (±BDNF 50 ng) in gastrocnemius, muscles were fixed, removed and serially sectioned. For each muscle, different serial sections were quantified for GFP-TTC fluorescence at the motor nerve terminals as described in material and methods. We found that the BDNF-dependent increase of GFP-TTC concentration at the NMJ, closely resembled that observed in LAL (1.51±0.12 fold increase vs 2.12±0.19 respectively).

### Example 11

### Influence of Other Neurotrophic Factors on GFP-TTC Localization at Motor Nerve Terminals

We also examined the effect of five additional trophic factors on GFP-TTC localization at the NMJ, including the neurotrophins NT-3; NT-4 and NGF as well as the neurocytokine CNTF (Ciliary Neurotrophic Factor), a member of the LIF cytokine family, and GDNF (Glial-Derived Neurotrophic Factor), a member of the TGF-β superfamily (Table 2). Many BDNF actions in neurons are mediated via the high affinity receptor tyrosine kinase TrkB, which is also the receptor for NT-4. Like BDNF, NT-4 also induced GFP-TTC localization at the NMJ (a 1.54±0.23 fold increase). A level of induction similar to NT-4 was also observed for GDNF (Table 2). On the other hand, even at high concentrations, neither NGF, NT-3, nor CNTF exhibited a significant effect on GFP-TTC localization.

TABLE 2
Effect of various neurotrophic factors on nerve terminal's
GFP-fluorescence level 30 min after in vivo GFP-TTC injection.

| | | Relative increase in |
|---|---|---|
| | Receptor | fluorescence level |
| BDNF | TrkB | 2.12 ± 0.19** |
| NT-4 | TrkB | 1.49 ± 0.23** |
| NT-3 | TrkC | 0.94 ± 0.05 |
| NGF | TrkA | 1.06 ± 0.06 |
| CNTF | CNTFRα | 0.95 ± 0.05 |
| GDNF | GFRα/cRET | 1.51 ± 0.02* |

GFP-TTC was co-injected with increasing concentrations of neurotrophic factors and GFP fluorescence quantified 30 min after as previously described. Mean of relative increase of GFP fluorescence of 2 or 3 independent experiments are indicated. Maximum fold induction was obtained for 50 ng of neurotrophic factor injected except for NT-3 (2.5 ng).
**P < 0.005;
*P < 0.05 t-test vs control.

### Example 12

### Comparison of Trk Receptors Distribution and GFP-TTC Localization at Motor Nerve Endings

Detection of either TrkB mRNA or protein in adult skeletal muscle and motoneurons has been reported in several studies (Funakoshi et al., 1993; Gonzalez et al., 1999; Griesbeck et al., 1995; Yan et al., 1997). Since our results indicated that the BDNF effect on GFP-TTC localization is dependent on TrkB receptor activation, it was of interest to determine whether GFP-TTC co-localized with TrkB at the NMJ of LAL muscles. Consistent with previous studies (Gonzalez et al., 1999; Sakuma et al., 2001), TrkB immunostaining was confined to the NMJ (FIG. 8). In the presynaptic side, TrkB staining was adjacent to, but not co-localized to the clusters of GFP-TTC labeling. Similar results were also obtained with an antibody that recognizes the activated Trk receptors (p-Trk, data not shown). This observation suggests that the mechanism whereby BDNF has an influence on the concentration of GFP-TTC at the nerve terminals, does not involve a direct interaction between TrkB and GFP-TTC or its receptors.

### Example 13

### Mechanisms Involved in BDNF Effect on GFP-TTC Concentration at the NMJ

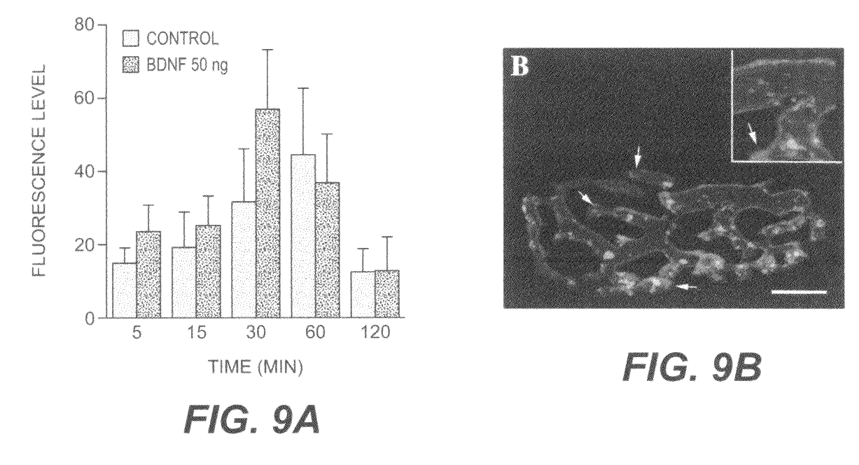

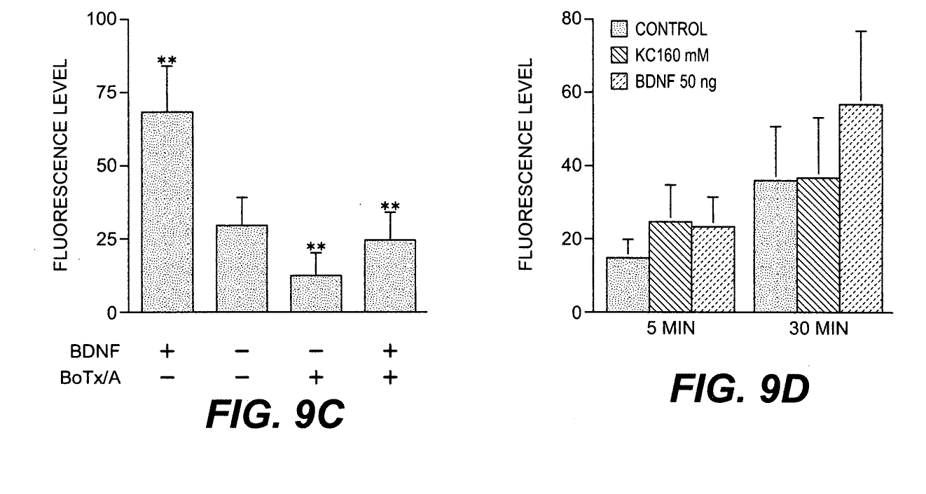

Possible explanations for the BDNF-induced enrichment of GFP-TTC at the NMJ could involve an elevated rate of localization of the probe at the NMJ, and/or an increased neuronal endocytosis of the probe. Confocal analysis performed 5, 15, 30, 60 and 120 min after GFP-TTC injection (±BDNF 50 ng) showed maximal labeling intensity at 30 min with BDNF injection, whereas in controls, the maximal staining occurred at 1 h and reached a level lower than that obtained with BDNF co-injection. After the first hour, similar levels of GFP-TTC were recorded at the NMJ in both conditions (FIG. 9A). These results are in accordance with previous results in Xenopus nerve-muscle co-culture indicating a time-limiting effect of BDNF (Lohof et al., 1993).

In vitro, tetanus neurotoxin internalization in neurons appears to involve both coated and uncoated-vesicular pathways (Herreros et al., 2001; Matteoli et al., 1996). Experiments performed either in vitro on excised LAL muscles with the endocytic fluid marker RH414 (data not shown), or immunostained against the SV2 synaptic vesicle proteins (FIG. 9B) and synaptophysin (data not shown) showed some overlapping with GFP-TTC labeling, indicating that the endocytosis of GFP-TTC was in part via recycling of neuronal synaptic vesicles. To differentiate between clathrin-dependent and clathrin-independent endocytic pathways, we used treatment with botulinum neurotoxin serotype A (BoTx/A), which blocks neurotransmitter release and endocytosis in motor nerve terminals (de Paiva et al., 1999). When BoTx/A was applied 48 hours before GFP-TTC injection, the probe level at the NMJ was markedly decreased by 50% (FIG. 9C), indicating that both clathrin-dependent and independent pathways are used to a comparable degree.

Enhanced synaptic transmission produced by application of exogenous BDNF; NT-3 or NT-4 involves a potentiation of neurotransmitter release (Lohof et al., 1993; Stoop and Poo, 1996; Wang and Poo, 1997). The increasing amount of GFP-TTC at the NMJ induced by BDNF injection could therefore be due in part to an elevated recycling of synaptic vesicles. To explore this hypothesis, increased exocytosis and endocytosis of synaptic vesicles were induced by GFP-TTC injection in a high-potassium medium. Five minutes after injection, exposure to high K⁺ medium or BDNF induced a similar increase of GFP-TTC level at the NMJ. However, after 30 min, the effect of high r was no longer detectable, whereas maximal induction was reached with BNDF at this time (FIG. 9D). Finally, even after neurotransmitter release and synaptic vesicle recycling were blocked by BoTx/A, an increased GFP-TTC signal was induced by BDNF treatment (FIG. 9C) with an amplitude comparable to that recorded in the non-paralyzed control NMJ (2.05 fold increase vs 2.12 respectively). Taken together, these results indicate that BDNF enhances an alternative endocytic pathway that appears to involve uncoated vesicles.

### Example 14

### Evidence for Association of GFP-TTC in Detergent-Insoluble Membrane at the Neuromuscular Junction

Binding of TTC to plasma membranes involves association to polysialogangliosides GD1b and GT1b, as well as a N-glycosylated 15 kDa protein. These three components partition preferentially in membrane microdomains called rafts. In vitro, TTC has been shown to associate with such microdomains in NGF-differentiated PC12 cells and in cultured spinal cord neurons (Herreros et al., 2001; Vyas, 2001). To test in vivo whether GFP-TTC associated to lipid rafts, gastrocnemius muscles were submitted to detergent extraction to isolate lipid microdomains after GFP-TTC intramuscular injection. Twelve fractions from the discontinuous sucrose gradient were collected and analyzed for distribution of GFP-TTC.

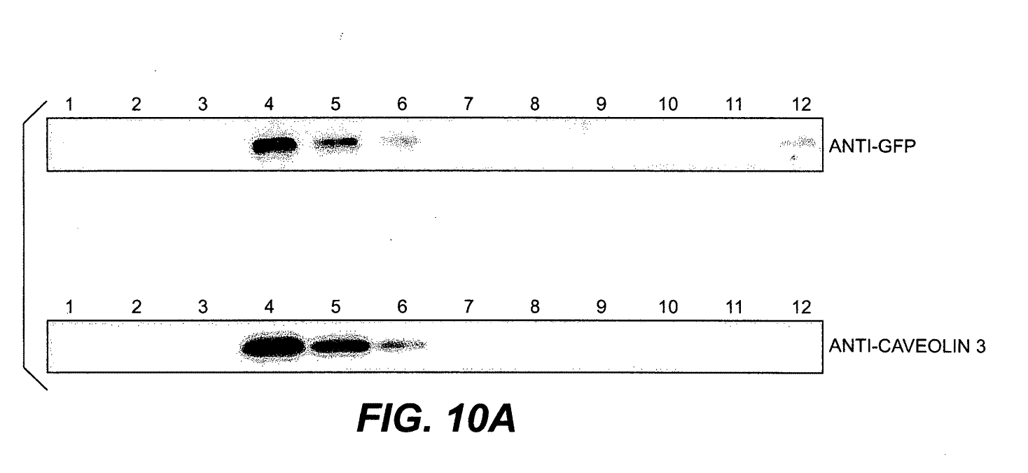

Neurons do not contain caveolin or morphologically distinct caveolae (Anderson, 1998), but significant fractions of cholesterol and glycosphingolipids are found in detergent-insoluble complexes, which are indistinguishable using the criteria of detergent insolubility from those associated with caveolae (Schnitzer et al., 1995). Thus, caveolin 3, a specific muscular caveolar marker (Tang et al., 1996), was used to identify the detergent-resistant fractions. Immunoblot analysis revealed that GFP-TTC co-migrated with raft microdomains, which contain caveolin 3 (FIG. 10A).

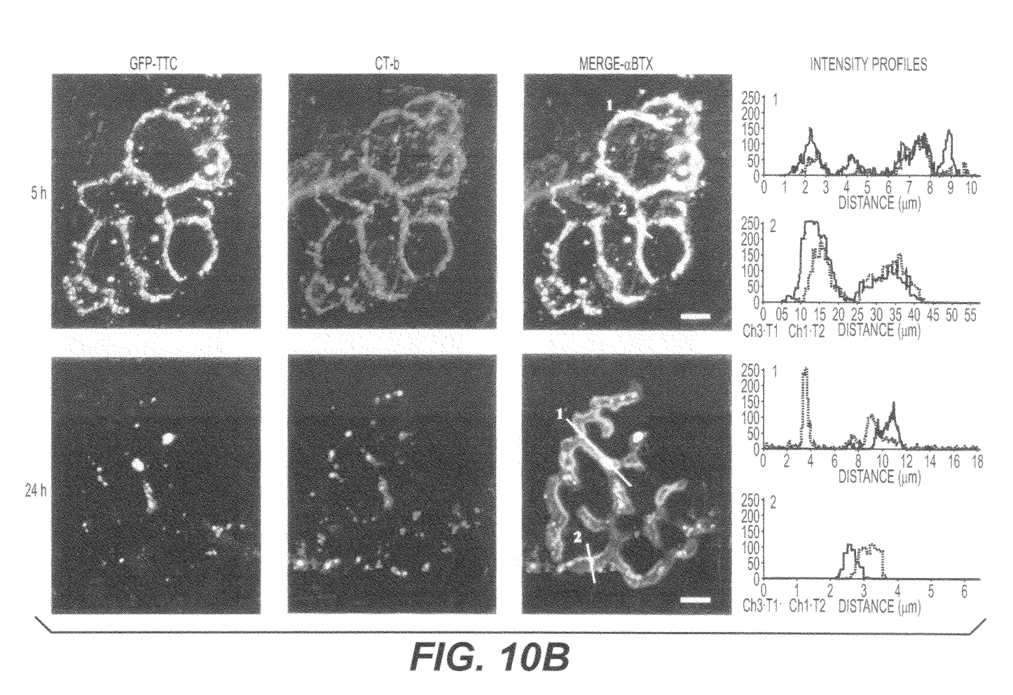

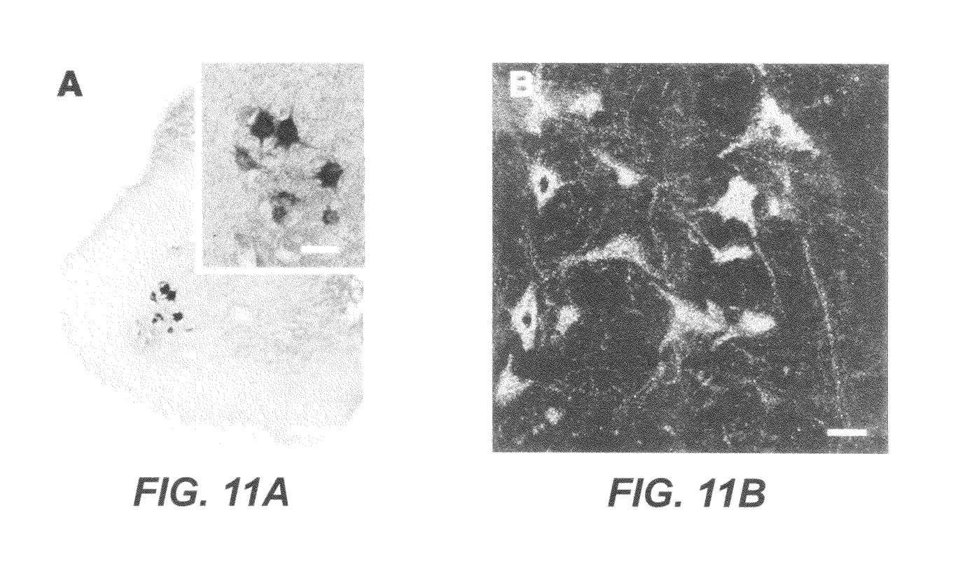

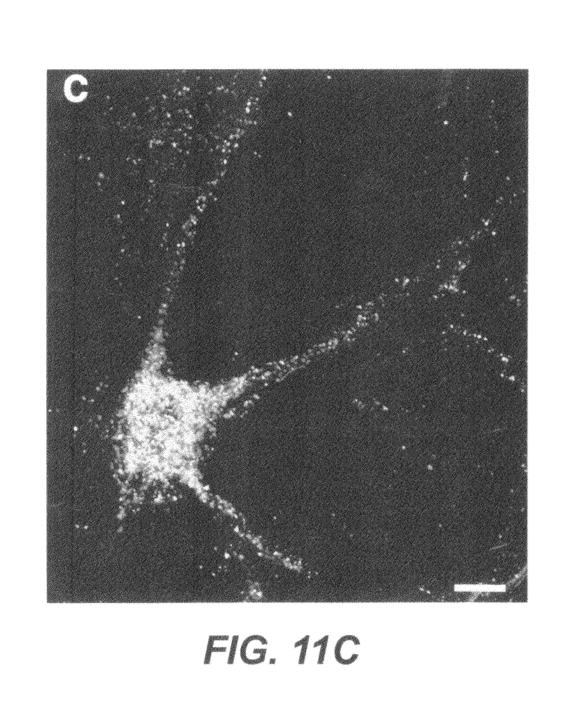

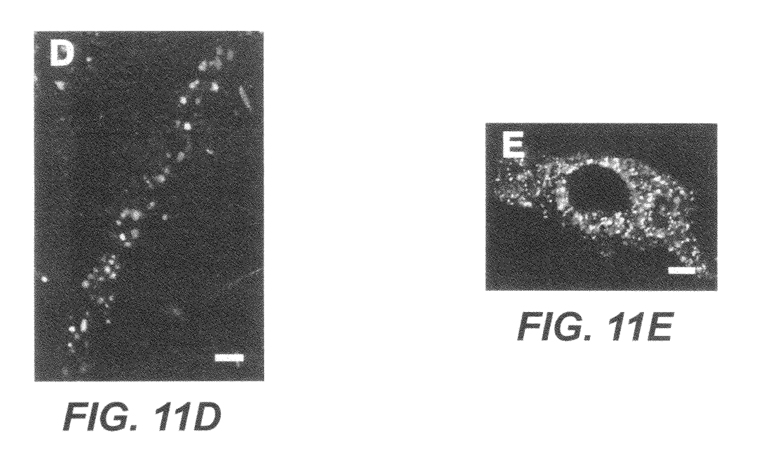

To investigate whether the GFP-TTC patches observed in vivo in motor nerve terminals correspond to lipid microdomains, we performed co-staining with Alexa 594-conjugated cholera toxin-B fragment (CT-b). CT-b specifically binds to ganglioside GM1, which is enriched in cholesterol-rich membrane microdomains, and is commonly used as a marker for membrane rafts and caveolae (Orlandi and Fishman, 1998; Schnitzer et al., 1995; Wolf et al., 1998). GFP-TTC and Alexa 594-conjugated CT-b fragment were co-injected into the gastrocnemius and confocal analysis was performed 1; 3; 5; 9 and 24 h later, with the NMJ being identified by AlexaFluor 647-conjugated α-BTX (FIG. 10B). Although the GFP-TTC labeling of motor nerve terminals was easily visualized in less than the 5 min necessary to process the tissue (FIGS. 6D and D′), CT-b was detectable at the NMJ only several hours after injection (3-5 h). Thus, the dynamics of trafficking of CT-b and TTC receptors to active synapse are clearly different. However, after 5 h, the distribution obtained for CT-b was similar to GFP-TTC staining, as characterized by diffuse staining and patches having an extensive overlap of the staining patterns obtained with GFP-TTC, indicating a localization of the TTC probe in lipid microdomains in motor nerve endings (FIG. 10B). Twenty four hours after gastrocnemius injection, both toxins had been internalized since only few patches, most of them positive for both toxins, persisted at the NMJ (FIGS. 10B and C). At this time, GFP-TTC and CT-b staining were detected in the same motoneuron cell bodies in the ventral horn of the spinal cord, but in different vesicular compartments (FIG. 11). Taken together, these results indicate that GFP-TTC used different lipid microdomains for neuronal binding and/or internalization pathways than CT-b.

### Materials and Methods

### Antibodies and Reagents.

Rabbit anti-GFP polyclonal antibodies were obtained from Invitrogen (1:5000 dilution). Mouse monoclonal antibody against caveolin 3 was from Transduction Laboratories (1:500). The monoclonal anti-neurofilament 200 (clone NE14) and the rabbit polyclonal anti-troponin T were obtained from Sigma. AlexaFluor 594-conjugated Cholera toxin subunit B (CT-b); AlexaFluor 488-conjugated goat-anti-rabbit IgG, AlexaFluor 647-conjugated α-bungarotoxin (α-BTX) and RH414 were obtained from Molecular Probes. Cy3-conjugated goat anti-rabbit IgG and Cy3-conjugated rat anti-mouse IgG were from Jackson Laboratories. TRITC-conjugated α-bungarotoxin was obtained from Calbiochem. The rabbit anti-TrkB (794) and the anti-p-Trk polyclonal antibody were obtained from Santa Cruz Technologies. The monoclonal antibody against synaptic vesicle protein SV2, developed by K. Buckley, was obtained from the Developmental Studies Hybridoma Bank developed under the auspices of the NICHD and maintained by The University of Iowa, Department of Biological Sciences, Iowa City. Monoclonal antibody against synaptic vesicle synaptophysin protein was obtained from Chemicon. The goat anti-rabbit and anti-mouse IgG antibodies conjugated to horseradish peroxidase were obtained from Pierce as well as the SuperSignal detection reagent. Recombinant neurotrophic factors rat CNTF; human NT-3; human NT-4, human BDNF, human GDNF and purified mouse NGF 7S were purchased from Alomone labs. Neurotrophic factors were prepared as stock solutions (10 μg/ml) and kept in aliquots at −80° C.

### In Vivo Intramuscular Injection.

All animal experiments were performed in accordance with French and European Community guidelines for laboratory animal handling. Six-week-old Swiss female mice were obtained from Charles River Breeding Laboratories. Intramuscular injections of β-gal-TTC, GFP-TTC fusion proteins, produced as previously described (Coen et al., 1997), or AlexaFluor 594-conjugated CT-b were intramuscular injected into the gastrocnemius muscle or subcutaneously in the immediate vicinity of the Levator auris longus (LAL) muscle on anesthetized mice. For fluorescence quantification, 25 μg of GFP-TTC fusion protein were injected in PBS in 50 μl final volume. For immunodetection or biochemical extraction, 50 μg of GFP-TTC probe were used. When co-injections with neurotrophic factors were performed, the volume injected was kept constant (50 μl). For injection in high K⁺, a physiological solution containing 60 mM KCl was co-injected with the probe.

### Botulinum Type-A Toxin Injection.

Clostridium botulinum type-A toxin (BoTx/A) was injected subcutaneously as a single dose of 0.05 ml containing about 0.5 μg of the purified neurotoxin in the vicinity of the LAL muscle of female Swiss mice (body weight 24-27 g). 48 h after BoTx/A treatment, a time sufficient for inducing muscle paralysis in the LAL due to blockade of neurotransmitter release (de Paiva et al., 1999), GFP-TTC (25 μg) was injected associated or not with BDNF (50 ng) in the vicinity of the LAL muscle. Mice were killed by intracardial injection of PFA 4% 30 min after injection and LAL muscle harvested and processed for confocal analysis.

### In Vitro Analysis of GFP-TTC Localization and Confocal Acquisition.

LAL muscles with their associated nerves were isolated from female Swiss-Webster mice (20-25 g), killed by dislocation of the cervical vertebrae. LAL muscles were mounted in Rhodorsil^{R}-lined organ baths (2 ml volume) superfused with a standard oxygenated physiological solution of the following composition (mM): NaCl-154; KCl 5; CaCl₂ 2; MgCl₂ 1; HEPES buffer 5 (pH=7.4) and glucose 11. Muscles were loaded for 45 min in the dark and at room temperature with both 25 μg GFP-TTC and 30 μM of RH414, dissolved in standard solution or, for synaptic vesicle recycling, in high K⁺ isotonic solution (with 60 mM KCl replacing 60 mM NaCl). Preparations were washed out of the GFP-TTC and RH414 dye, and rinsed several times with dye-free standard medium before being imaged with a Leica TCS SP2 confocal laser scanning microscope system (Leica Microsystems, Germany) mounted on a Leica DM-RXA2 upright microscope equipped with a ×40 water immersion lens (Leica, NA 0.8). The confocal system was controlled through Leica-supplied software running on a Windows NT workstation.

### Preparation of Detergent-Resistant Membrane (DRMs) Fractions and Western Blot

Preparation of Detergent-Resistant Membrane Fractions is One of the Most widely used methods for studying lipid rafts. Two hours after GFP-TTC injection (50 μg), mouse gastrocnemius muscle tissue was harvested, minced with scissors and homogenized in 2 ml of MES-buffered saline containing 1% (v/v) Triton X-100. Homogenization was carried out with a Polytron tissue grinder. After centrifugation at low speed for 5 min, supernatant was adjusted to 40% sucrose. A 5-30% linear sucrose gradient was formed above the homogenate and centrifuged at 39,000 rpm for 18 h in a SW41 rotor. Then, 11-12 fractions of 1 ml were collected from the top of the gradient and precipitated with 6.5% trichloroacetic acid in the presence of 0.05% sodium deoxycholate and washed with 80% cold acetone. Samples were analyzed by Western Blot after separating on a 4-15% SDS-PAGE followed by Western Blot. Membranes were probed first with polyclonal anti-GFP and monoclonal anti-caveolin 3 antibodies, and then incubated with goat anti-rabbit IgGs and goat anti-mouse IgGs antibodies conjugated with horseradish peroxidase. The SuperSignal (Pierce) was used to visualize the reaction

### Quantification of GFP-TTC Fluorescence Intensity at the NMJ

After intracardiac perfusion and fixation, LAL muscles were harvested, washed in PBS for 20 min, stained with TRITC-conjugated α-bungarotoxin (TRITC-a-BTX) (2 μg/ml) for 45 min at 37° C. in PBS and washed twice in PBS. Images were acquired on an Axiovert 200M laser scanning confocal microscope (LSM-510 Zeiss; version 3.2) through a ×40/1.2 water-immersion objective using LP560 and BP505-550 filters. The pinhole aperture was set to 1 airy unit, and images were digitized at 8-bit resolution into a 512×512 pixel array. To be able to compare the intensity of GFP staining between different experiments, laser illumination, photomultiplier gain in regard of linear response, and other acquisition variables were standardized. To quantify GFP-TTC localization at the NMJ, series of “look-through” projection (of MIP: Maximum Intensity Projection) was generated. Images from each NMJ were processed identically: NMJ surface area (in μm²) was determined by TRITC-a-BTX labeling and GFP fluorescence global intensity (sum of each pixel intensity) was then measured only in this predefined area. This value, divided by the NMJ area yielded GFP fluorescence intensity per square micrometer, which thus defined the fluorescence level expressed as arbitrary units. For each condition, ˜15 to 20 synapses were quantified and results were expressed as the mean±SD. Statistical significance was defined as p<0.05 using a two-tailed t test. Each experiment was repeated at least two or three times.

### Analysis of Spinal Cord.

24 hours after β-gal-TTC or GFP-TTC and CT-b injection into the gastrocnemius muscle (50 μg each), mice deeply anesthetized were perfused intracardially with 4% PFA. The spinal cord was harvested and embedded in Tissue Tek embedding media after overnight incubation in 25% sucrose in PBS 0.1 M. Longitudinal cryostat sections (30 μm thickness) were cut and mounted onto coated slides.

### X-gal Reaction.

X-gal reaction was performed as previously described (Coen et al., 1997).

Further work was carried out relating to fusion proteins composed of the atoxic C-terminal fragment of tetanus toxin (TTC) and green fluorescent protein or β-galactosidase (GFP-TTC and β-gal-TTC, respectively), that rapidly cluster at motor nerve terminals of the mouse neuromuscular junction (NMJ). Furthermore, this trafficking of GFP-TTC and β-gal-TTC fusion proteins involves presynaptic activity, via the secretion of active molecules, including being affected by brain-derived neurotrophic factor (BDNF). Quantitative confocal microscopy and a fluorometric assay for β-gal activity can be used to reveal that co-injecting BDNF and the TTC fusion proteins significantly increases the kinetics and amount of the proteins' localization at the NMJ and their internalization by motor nerve terminals. In one aspect of the invention, these observed increases are independent of synaptic vesicle recycling, because BDNF does not affect spontaneous quantal acetylcholine release. In another aspect of the invention, the injection of anti-BDNF antibody shortly before the injection of GFP-TTC, and before the co-injection of GFP-TTC and BDNF, significantly reduces the fusion protein's localization at the NMJ. In addition, the co-injection of GFP-TTC with neurotrophin-4 (NT-4) or glial-derived neurotrophic factor (GDNF), but not with nerve growth factor, neurotrophin-3 or ciliary neurotrophic factor, can also significantly increase the fusion protein's localization at the NMJ. Thus, the TTC fusion proteins may use for their neuronal internalization endocytic pathways normally stimulated by BDNF, NT-4 and GDNF. Different tyrosine kinase receptors with similar signaling pathways are activated by BDNF/NT-4 and GDNF through their respective receptors. Thus, activated components of BDNF/NT-4 and GDNF signaling pathways can be involved in the internalization of TTC fusion proteins. In one aspect of the invention, the internalization of TTC fusion proteins can be facilitated by the localization of TTC receptors in specific membrane microdomains or by recruiting various factors needed for internalization of TTC.

More generally, the atoxic C-terminal fragment of tetanus toxin (designated ‘TTC’) is efficiently internalized by nerve endings and is transported retrogradely along axons to the spinal cord. The fragment's in vivo internalization and transport is maintained even when it is fused to a reporter gene such as LacZ, which encodes for β-galactosidase (β-gal) activity, or to green fluorescent protein (GFP), either after direct injection of the hybrid protein (Coen et al., 1997; Miana-Mena et al., 2003; Sapir et al., 2004) or when expressed as a transgene in mice (Maskos et al., 2002; Miana-Mena et al., 2004; Sakurai et al., 2005). Roux et al. (2005) previously reported that TTC fusion proteins localize quickly, via clathrin-coated pits and axolemmal infoldings associated with lipid microdomains, in nerve terminals of the mouse neuromuscular junction (NMJ). Moreover, in cultured neuronal cells, evidence has been presented for a co-trafficking between TTC and the p75 neurotrophin receptor (p75^{NTR}) (Lalli & Schiavo, 2002). Furthermore, tetanus toxin and TTC mediate activation of intracellular pathways involving neurotrophin tyrosine kinase receptors (Trk) (Gil et al., 2003; Chaïb-Oukadour et al., 2004). Thus, these results suggest a relationship between TTC trafficking, neurotrophins and neurotrophin receptors.

Neuronal activity influences the synthesis, release and effectiveness of neurotrophins (Funakoshi et al., 1995; Wang & Poo, 1997; Xie et al., 1997; Gomez-Pinilla et al., 2001). Thus, presynaptic depolarization greatly facilitates modulation of synaptic transmission at developing NMJs by brain-derived neurotrophic factor (BDNF) (Boulanger & Poo, 1999). Also, high-frequency neuronal activity and synaptic transmission have been found (Du et al., 2000) to elevate the number of specific tyrosine kinase receptors of BDNF (TrkB) on the surface of cultured hippocampal neurons, apparently by recruiting extra TrkB receptors to the plasma membrane (Meyer-Franke et al., 1998). Neuronal activity also influences the rapid clustering of TTC probes at the NMJ (Miana-Mena et al., 2002), possibly as a result of activity-dependent secretion or action of various signaling molecules at the synapse, or both. One such neurotrophic factor, BDNF, can modulate in vivo localization and internalization of TTC in motor nerve terminals of mature mouse NMJs.

Confocal microscopy and fluorometric measurement of β-gal activity revealed that BDNF that has been co-injected with GFP-TTC or β-gal-TTC significantly increased the fusion proteins' localization and internalization at motor nerve endings. Similar results were obtained after co-injection with neurotrophin-4 (NT-4), also a TrkB ligand, which suggests that the TrkB receptor has a role in TTC trafficking, even though the receptor localizes in membrane domains that are distinct from those containing GFP-TTC in nerve terminals of the NMJ. In addition, endogenous BDNF can also be involved in the neuronal internalization of TTC. Thus, endogenous BDNF can participate in the mechanism that modulates the transport of TTC in a functional neuronal network.

The influence of the neurotrophic factor BDNF on motor nerve terminal internalization and retrograde axonal transport of β-gal-TTC and GFP-TTC fusion proteins has been further investigated. Co-injecting BDNF and GFP-TTC significantly enhances localization of GFP-TTC in the nerve terminals at the NMJ, as demonstrated by their increased fluorescence. The response is detectable as early as 5 min post-injection, reaches a maximum at 30 min post-injection, and is blocked by an anti-BDNF antibody. The antibody also inhibits the ability of endogenous BDNF to facilitate the fusion protein's localization in motor nerve endings. Because GFP-TTC localizes in motor nerve terminals (Roux et al., 2005), it is likely that BDNF increases its presynaptic internalization. This idea was confirmed by the detection of a significant increase in the sciatic nerve's β-gal activity after co-injecting BDNF and β-gal-TTC into the gastrocnemius muscle. BDNF significantly facilitates localization of β-gal-TTC in the sciatic nerve; however, it does not appear to act by enhancing the fusion protein's retrograde axonal transport (Tables 3 and 5, respectively). This result is in accordance with that of a previous study (Sagot et al., 1998) examining the influence of neurotrophic factors on retrolabeling of motoneurons.

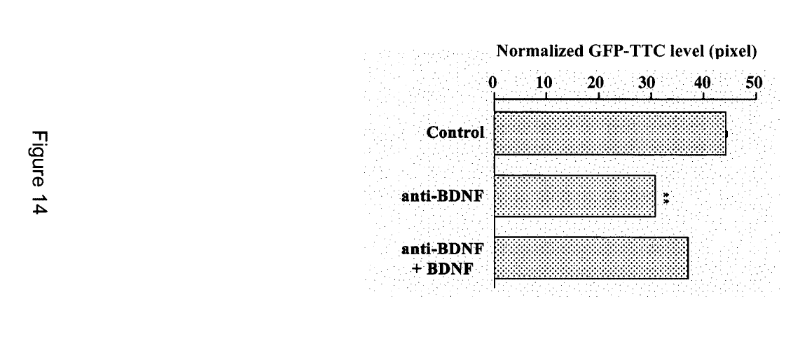

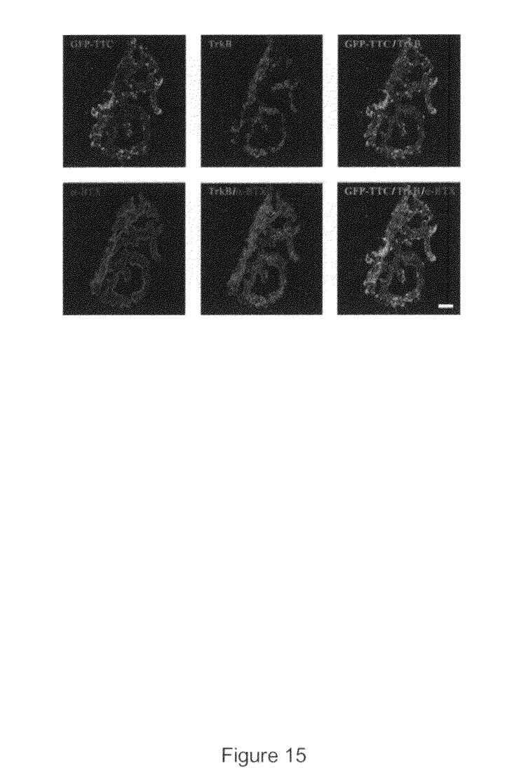

Neurotrophins exert their effects by interacting with two structurally unrelated transmembrane receptors: p75^{NTR}, a member of the tumor necrosis factor receptor superfamily (Dechant & Barde, 1997), and the Trk tyrosine kinase receptors (Barbacid, 1995; Bothwell, 1995). The p75^{NTR} receptor binds to all neurotrophins with equivalent affinity; however, the neurotrophins' interaction with Trk receptors is specific (Ip et al., 1993). Thus, BDNF and NT-4 bind preferentially to TrkB, NGF to TrkA, and NT-3 to TrkC (Barbacid, 1995; Bothwell, 1995). NT-4, like BDNF, increases the GFP-TTC probe's concentration in nerve terminals (Table 5); therefore, it is likely that the mechanism by which they enhance localization of TTC involves TrkB activation. However, specific co-localization between GFP-TTC and TrkB or p-Trk receptors is not detected at the NMJ under control conditions (FIG. 15) or after co-injecting BDNF and GFP-TTC. Therefore, it appears unlikely that GFP-TTC and TrkB localize in a similar endocytic compartment. Recently, Pincher-dependent macropinocytosis of TrkB, involving Pincher-positive tubular structures that surrounded cytoplasmic accumulations of p-TrkB, was described in sympathetic and hippocampal neurons (Valdez et al., 2005). The tubular structures may be related to the nerve terminal axolemma infolding involved in the internalization of TTC (Roux et al., 2005); however, complementary experiments are required to evaluate that possibility. The concentration of GFP-TTC in nerve terminals is in accordance with recent results indicating that in vivo transport of TTC is similar, but not identical, to that of some neurotrophic factors. Thus, although p-TrkB and β-gal-TTC localize in axonal large uncoated vesicles (Bhattacharyya et al., 2002; Miana-Mena et al., 2002), TTC uses a pathway that does not require a multivesicular body to transfer to dendrites, unlike BDNF and GDNF that are also retrogradely and trans-synaptically transported, but accumulate at synapses more slowly (Rind et al., 2005). A distinct internalization and retrograde axonal pathway for p75^{NTR} and TrkA has been described (Bronfman et al., 2003) in PC12 cells. This unique pathway may explain why localization of TTC and TrkB differs in vivo; whereas, in cultured motoneurons, TTC localizes in the same retrograde transport organelles as p75^{NTR} (Lalli & Schiavo, 2002).

In contrast to BDNF and NT-4, NGF and NT-3 do not significantly affect nerve terminal internalization of GFP-TTC (Table 4). Motoneurons are known to express only TrkB and TrkC (Funakoshi et al., 1993; Yan et al., 1993; Ehlers et al., 1995), which do not serve as the primary receptors for NT-4, NGF and NT-3, perhaps explaining the lack of effect of NGF on GFP-TTC localization at NMJ. Although, NT-3 also stimulates TrkB, although to a lesser extent than does BDNF (Barbacid, 1995). However, even after the injecting a relatively large dose (250 ng) of NT-3, enhancement of the TTC fusion protein's internalization can not be detected. The inactivity of NT-3 in our model may be explained by the fact that the responses generated by TrkB stimulation depend, in part, on which ligand it binds (Fan et al., 2000). Moreover, although Trk receptors activate three similar signaling pathways; i.e. the phosphatidylinositol (PI)₃ kinase, the Ras-MAP kinase-dependent, and the PLCγ (phospholipase C) signaling pathways (reviewed by Segal & Greenberg, 1996; Huang & Reichardt, 2003), differences in signaling exist, which may explain the opposite effects of NT-3 and BDNF (Munson et al., 1997; Mendell et al., 1999; Seebach et al., 1999). Thus, it is possible that, under the conditions of our experimental protocols, NT-3 binding is not able to elicit the response(s) required for GFP-TTC internalization. Finally, there is a possibility that the LAL muscle is more sensitive to BDNF/NT-4 than to NT-3, in accordance with reports from, Klein et al., 1993; Koliatsos et al., 1994; and Silos-Santiago et al., 1997, which suggest a physiological role for TrkB and the ligands BDNF and NT-4 in the facial nerve nucleus, which innervates the LAL muscle.

GDNF also significantly increases GFP-TTC localization at the mature mouse NMJ (Table 4). However, in contrast to BDNF, GDNF has been reported to enhance spontaneous quantal transmitter release by neonatal mouse NMJs (Ribchester et al., 1998) and in Xenopus nerve-muscle co-cultures (Stoop & Poo, 1996; Liou et al., 1997). Thus, the possibility that GDNF facilitates GFP-TTC internalization by increasing synaptic vesicle recycling can not be ruled out. After binding to a GPI-anchored co-receptor (GDNF family receptor-α; GFRα), GDNF signals via the RET tyrosine kinase receptor (Durbec et al., 1996; Trupp et al., 1996) that activates the same signaling pathway as TrkB (Takahashi, 2001; Airaksinen & Saarma, 2002). Interestingly, although CNTF does not enhance the internalization of GFP-TTC at the NMJ (Table 4), it activates distinct signaling pathways, via the associated cytoplasmic Jak tyrosine kinase receptor, after binding to its receptor (Segal & Greenberg, 1996). Therefore, it is likely that various signaling pathways activated by distinct neurotrophic factors are involved in facilitating the internalization of TTC.

Several pathogens activate the MAP kinase pathway in order to gain entry into cells (Tang et al., 1998; Liu et al., 2002). TTC has been reported (Gil et al., 2000, 2001, 2003; Chaïb-Oukadour et al., 2004) to activate intracellular pathways involving Trk receptors, and Gil et al. (2003) have proposed that stimulation of Trk receptors by tetanus toxin (TeNT) is involved in the toxin's transport to the CNS. In addition, exogenous GT1b and GD1b, which bind TeNT and TTC (Halpern & Neale, 1995), activate phosphorylation of Trk and Erk1/2 (Duchemin et al., 2002). Moreover, changes in the endogenous GM1 ganglioside density in the TrkB receptor's environment affect basal or BDNF-induced receptor activity (Pitto et al., 1998). Thus, it is possible that binding of TTC to its receptors induces their redistribution in specific membrane domains located near TrkB receptors, thus facilitating the TrkB activation that is necessary for TTC internalization. Conversely, TrkB activation may cause re-localization of membrane domains containing TTC receptors, thereby facilitating neuronal internalization of TTC. TTC receptors (Herreros et al., 2001; Vyas et al., 2001) and TrkB (Wu et al., 1997) are highly enriched in lipid microdomains of neuronal plasma membranes, but there is no direct link in the binding of BDNF and TTC. Lipid rafts are cholesterol- and sphingolipid-rich lipid microdomains in eukaryotic cell membranes (Simons & Ikonen, 1997), and they are believed to function in neuronal signaling by concentrating or separating specific molecules in a unique lipid environment (Simons & Toomre, 2000; Galbiati et al., 2001). For example, the co-receptor GFRα1, which binds GDNF, localizes in lipid rafts and recruits c-RET to lipid rafts after GDNF stimulation (Tansey et al., 2000; Paratcha et al., 2001). Also, activation of PKA is required to translocate activated p75^{NTR} to lipid rafts (Higuchi et al., 2003). Moreover, many of the intermediates in the signaling cascades activated by BDNF binding to its cognate receptor are also present in lipid rafts (Simons & Toomre, 2000; Tsui-Pierchala et al., 2002). Therefore, because BDNF, NT-4 and GDNF activate different receptors, we now propose that a component(s) of their activation pathways activates the trafficking of TTC receptors in specific lipid microdomains, probably by facilitating the recruitment of factors needed for the toxin fragment's internalization.

Thus, in one aspect of the invention, the internalization of GFP-TTC and β-gal-TTC fusion proteins is regulated by neurotrophic factors, such as the stimulation of constitutive endocytic pathways by BDNF, via TrkB activation.

### ADDITIONAL EXAMPLES

### Example 15

### BDNF Enhances GFP-TTC Localization in Motor Nerve Terminals

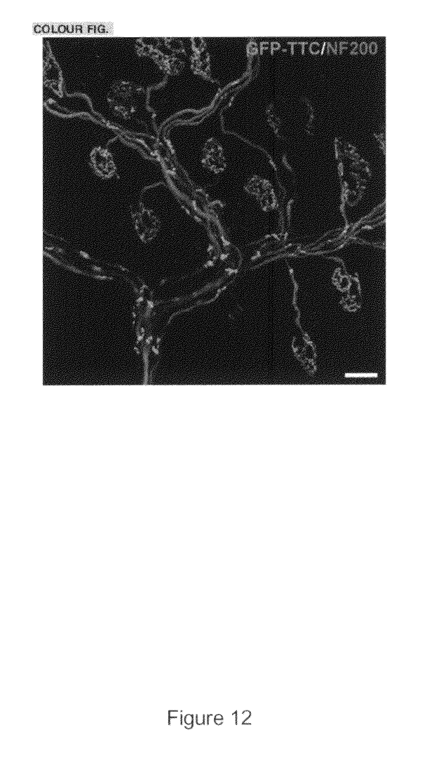

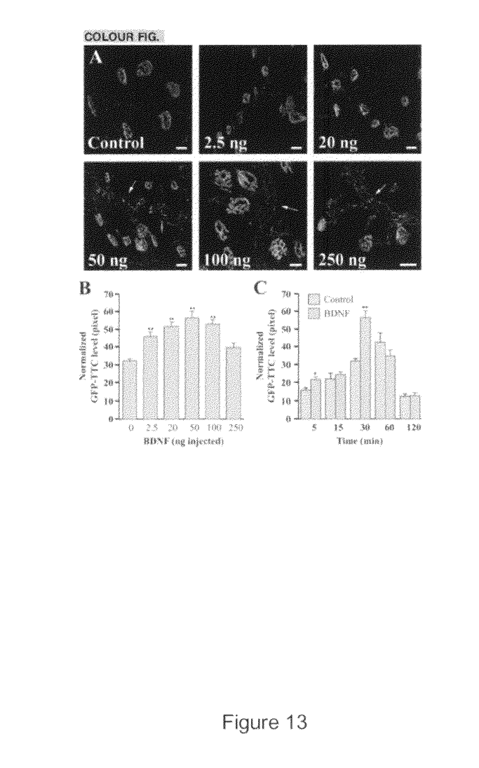

To assess whether exogenously applied BDNF affects GFP-TTC recruitment in motor nerve terminals, various concentrations of this neurotrophin (2.5-250 ng) were co-injected with GFP-TTC in the immediate vicinity of the LAL muscle, and control mice were injected only with GFP-TTC. The LAL muscle is a thin and flat Muscle consisting of only a few layers of fibers; therefore, whole-mount neuromuscular preparations could be easily examined by confocal microscopy (FIG. 12). Mice were killed 30 min. post-injection, the LAL muscles were removed, and the NMJs were identified by probing with AlexaFluor 594-conjugated α-bungarotoxin (α-BTX) (FIG. 13A). Quantitative determination of GFP-TTC levels revealed that BDNF elicited a significant, concentration-dependent enhancement of GFP-TTC fluorescence at the NMJ. The most pronounced effect (2.12±0.19-fold increase) was observed with 50 ng of BDNF (FIG. 13B); i.e. higher doses (100 and 250 ng) elicited weaker elevations of GFP-TTC localization at the NMJ (1.72±0.12- and 1.15±0.22-fold, respectively). Roux et al. (2005) previously reported that GFP-TTC localizes in nerve terminals; therefore, it is likely that BDNF increases the fusion protein's presynaptic localization.

In order to exclude the possibility that the effect of BDNF is specific for the LAL muscle, we examined its ability to affect the localization of GFP-TTC in a different muscle. Therefore, gastrocnemius muscles were injected with a mixture of GFP-TTC and BDNF (50 ng) or with GFP-TTC alone (control). The muscles were fixed by intracardiac perfusion with PFA (30 min post-injection), removed from the mice and serially sectioned, and the GFP-TTC-mediated fluorescence of the section at the motor nerve terminals was quantified as described in Materials and methods. BDNF significantly increased the amount of GFP-TTC in gastrocnemius muscle junctions (1.51 ±0.12-fold). Thus, we can conclude that the effect of BDNF on GFP-TTC concentration at the NMJ is not limited to the LAL, but also occurs in the gastrocnemius muscle.

AlexaFluor 594-conjugated or AlexaFluor 647-conjugated α-bungarotoxin (α-BTX) from Molecular Probes Europe BV (Leiden, the Netherlands). Human BDNF, was purchased from Alomone Laboratories (Jerusalem, Israel), and was prepared as a stock solution (10 μg/mL). Aliquots were stored at ±80° C. until used.

All animal experiments were performed in accordance with French and European Community guidelines for laboratory animal handling. Six-week-old Swiss female mice were obtained from Charles River Breeding Laboratories (L'Arbresle, France), and they were deeply anaesthetized, by intraperitoneal (i.p.) injection of sodium pentobarbital (90 mg/kg body weight), during all procedures. GFP-TTC (Kissa et al., 2002), produced as previously described (Coen et al., 1997), was injected subcutaneously (s.c.) in the immediate vicinity of the levator auris longus (LAL) muscle, or intramuscularly (i.m.) into the gastrocnemius muscle.

GFP-TTC fluorescence at the NMJ was quantified after fixation by intracardiac perfusion with freshly prepared paraformaldehyde (PFA, 4%), GFP-TTC-treated [25 μg in 50 μL of 0.1 m phosphate-buffered saline (PBS)] and control LAL muscles were harvested, washed with PBS for 20 min, incubated/probed (45 min, 37° C.) with α-BTX (2 μg/mL PBS) and washed twice in PBS. Images were acquired with an Axiovert 200M laser scanning confocal microscope system (LSM-510 Zeiss, version 3.2, Gottingen, Germany) equipped with a ×40/1.2 water-immersion objective and with LP560 and BP505-550 filters. The pinhole aperture was set to 1 airy unit, and images were digitized (8-bit resolution) into a 512×512 pixel array. In order to compare the intensity of GFP staining obtained during different experiments, laser illumination, the photomultiplier gain's linear response and other acquisition parameters were standardized. To quantify GFP-TTC at the NMJ, a series of look-through' projections were generated in order to determine the maximum intensity projection. Images from each NMJ were processed identically, and the surface area of the NMJ (S_{BTX}, in μm²) was determined by α-BTX labeling. The GFP fluorescence global intensity (F, the sum of each pixel intensity) and GFP-labeled surface area (S_{GFP}, in μm²) subsequently were measured only in this predefined area. F×S_{GFP} yielded the amount of GFP-TTC located at the NMJ, and dividing this value by the area of the NMJ (S_{BTX}) yielded the normalized GFP-TTC level for the area of the NMJ. Fifteen-20 junctions were quantified for each condition, and the results were expressed as the mean±SEM. Statistical significance was defined as P<0.05, using the two-tailed Student's t-test. Each experiment was performed at least in triplicate. For fluorescence quantification at the NMJs of gastrocnemius muscle cryosections were performed. Briefly, 30 min after GFP-TTC i.m. injection, muscles were fixed, incubated overnight in PBS (supplemented with 25% sucrose) and embedded in OCT. Cryostat serial sections (30 μm) were processed, probed with α-BTX and analyzed as described above.

GFP-TTC fluorescence at the NMJ was quantified after fixation by intracardiac perfusion with freshly prepared paraformaldehyde (PFA, 4%), GFP-TTC-treated [25 μg in 50 μL of 0.1 M phosphate-buffered saline (PBS)] and control LAL muscles were harvested, washed with PBS for 20 min, incubated/probed (45 min., 37° C.) with α-BTX (2 μg/mL PBS) and washed twice in PBS. Images were acquired with an Axiovert 200M laser scanning confocal microscope system (LSM-510 Zeiss, version 3.2, Göttingen, Germany) equipped with a ×40/1.2 water-immersion objective and with LP560 and BP505-550 filters. The pinhole aperture was set to 1 airy unit, and images were digitized (8-bit resolution) into a 512×512 pixel array. In order to compare the intensity of GFP staining obtained during different experiments, laser illumination, the photomultiplier gain's linear response and other acquisition parameters were standardized. To quantify GFP-TTC at the NMJ, a series of look-through' projections were generated in order to determine the maximum intensity projection. Images from each NMJ were processed identically, and the surface area of the NMJ (S_{BTX}, in μm²) was determined by α-BTX labeling. The GFP fluorescence global intensity (F, the sum of each pixel intensity) and GFP-labeled surface area (S_{GFP}, in μm²) subsequently were measured only in this predefined area. F×S_{GFP} yielded the amount of GFP-TTC located at the NMJ, and dividing this value by the area of the NMJ (S_{BTX}) yielded the normalized GFP-TTC level for the area of the NMJ. Fifteen-20 junctions were quantified for each condition, and the results were expressed as the mean±SEM. Statistical significance was defined as P<0.05, using the two-tailed Student's t-test. Each experiment was performed at least in triplicate. For fluorescence quantification at the NMJs of gastrocnemius muscle cryosections were performed. Briefly, 30 min after GFP-TTC i.m. injection, muscles were fixed, incubated overnight in PBS (supplemented with 25% sucrose) and embedded in O.C.T. Cryostat serial sections (30 μm) were processed, probed with α-BTX and analyzed as described above.

### Example 16

### BDNF Increases Neuronal Internalization of β-gal-TTC at the NMJ

To confirm that BDNF enhances the internalization of TTC fusion proteins, experiments were performed with a β-gal-TTC probe, because β-gal activity is easily quantified with a fluorimetric assay (Forlani & Nicolas, 1996). The β-gal activity detected in a sciatic nerve that was ligated just before co-injecting the gastrocnemius muscle with β-gal-TTC and BDNF was significantly greater than when the muscle was injected with β-gal-TTC alone (Table 3). BDNF-mediated enhancement of β-gal-TTC localization in the sciatic nerve was similar to that observed at NMJs of LAL muscles co-injected with BDNF and GFP-TTC (2.44±0.08-fold and 2.12±0.19-fold increases, respectively), thus confirming that BDNF significantly increases TTC internalization by motor nerve terminals. Also, as previously observed for the NMJs of LAL muscles injected with GFP-TTC, the effect observed was higher with 50 ng than with 5 ng of BDNF (Table 1). β-gal-TTC (Coen et al., 1997), produced as previously described (Coen et al, 1997), was injected subcutaneously (s.c.) in the immediate vicinity of the gastrocnemius muscle.

The sciatic nerve was ligated, by first anaesthetizing the mouse, exposing one of the sciatic nerves, and ligating the nerve approximately 1 cm proximal to the gastrocnemius muscle (i.e. towards the body axis). Two ligatures (surgical 5.0-silk sutures) were placed adjacent to each other on the sciatic nerve and the wound was closed.

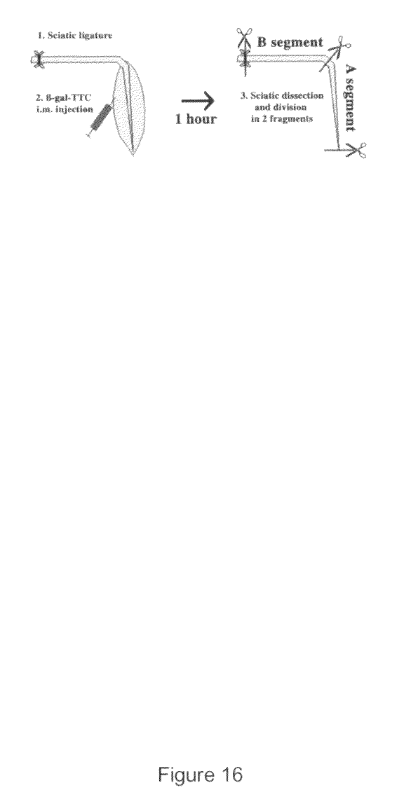

Immediately after ligating the sciatic nerve, the gastrocnemius muscles of the test mice were injected with PBS (50 μL) containing β-gal-TTC (50 μg) and BDNF (5 or 50 ng). The control mice were injected with the fusion protein but not with BDNF. One hour post-injection, the mice were deeply anaesthetized and intracardially perfused with PBS to eliminate blood, and the sciatic nerves were dissected, harvested (on ice) and divided into two segments. The segment corresponding to the nerve branch entering the gastrocnemius muscle was designated the “A-segment,” and the segment corresponding to the portion of the nerve close to the ligature was designated the “B-segment” (FIG. 16). The nerve tissue was disrupted by sanitation in Tris-buffered saline (pH=7.4) supplemented with 15% glycerol, 10 mm dithiothreitol, 0.01% Nonidet P-40 and 0.05% sodium deoxycholatate, and the suspensions were centrifuged (13 000 g, 10 min, 4° C.) in a Hereaus Biofuge 13 (Hereaus Sepatech, Osterode/Harz, Germany). The protein concentration of the supernatant fluids was determined by the Bradford method (as described by Bio-Rad Laboratories, Richmond, Calif., USA), and their β-gal activity was quantified by the fluorogenic substrate [4-methylumbelliferyl β-d-galactoside (MUG)] method described by Forlani & Nicolas (1996). Briefly: (i) aliquots of the supernatant fluids were transferred into individual wells of a 96-well microtitre plate; (ii) MUG buffer [pH 7.2, and composed of (in mm): Na₂HPO₄, 60; NaH₂PO₄, 40; KCl, 10; MgSO₄, 1; Triton X-100, 0.5%; sodium azide, 0.1%; bovine albumin, 100 μg/mL; MUG, 44] was added to start the reaction; and (iii) the mixtures were incubated for 2 h at 37° C. At various times during the incubation, fluorescence measurements were made with a Fluoroscan Ascent fluorimetric plate reader equipped with computer-controlled software (Fluoroscan Ascent Thermo; Electron Corporation, Vantaa, Finland), and the number of international units (IU) of β-gal activity was evaluated.

TABLE 3
The effect of BDNF on internalization of β-gal-TTC by the sciatic nerve

| | | β-gal activity |
|---|---|---|
| | β-gal activity | internalized/injected activity |
| | internalized (IU) | (%) |
| Control | 9707.977 ± 11132.248 | 6.35 ± 0.74% |
| BDNF 5 ng | 14 123.880 ± 1612.392 | 9.24 ± 1.06% |
| BDNF 50 ng | 21 857.918 ± 556.137 | 14.31 ± 0.36% |

β-gal-TTC and BDNF (5 or 50 ng) were co-injected into the gastrocnemius muscle just after sciatic nerve ligation. One hour post-injection, the sciatic nerve was harvested and separated into two segments, the A-segment which was proximal to the gastrocnemius muscle, and the B-segment, which was proximal to the nerve ligature (FIG. 16). The nerve tissue was disrupted by sanitation, and its β-gal activity was measured by a fluorimetric assay in each segment. The mean of internalized enzymatic activity in sciatic nerve (the β-gal activity in the A-segment + the β-gal activity in the B-segment) ± SD of three independent experiments was expressed in global β-gal activity in IU or as a percentage of the injected activity (1.5 × 10⁹ IU).

### Example 17

### BDNF Increases the Kinetics of GFP-TTC Internalization by Motor Nerve Endings

In order to determine whether BDNF could modify the kinetics of GFP-TTC internalization by motor nerve endings, we performed confocal analysis at various times (5, 15, 30, 60 and 120 min) after injecting LAL muscles with GFP-TTC (controls) and after co-injecting them with BDNF (50 ng) and GFP-TTC (FIG. 13C). Under control conditions, maximal staining occurred 60 min. post-injection; however, after co-injecting BDNF and GFP-TTC, maximal labeling intensity was observed 30 min post-injection, and it reached a higher average level than that in the control muscles. After 60 min. post-injection, similar GFP-TTC levels were recorded at the NMJs of control muscles and muscles co-injected with BDNF and GFP-TTC. These results were in accordance with previous results (Lohof et al., 1993) obtained with Xenopus nerve-muscle co-cultures, which indicate a time-limited effect of BDNF. Taken together, these results indicate that BNDF increases the rate of concentration of GFP-TTC at motor nerve endings.

### Example 18

### In Vivo Neutralization of BDNF Decreases GFP-TTC Localization at Motor Nerve Endings

To test the contribution of endogenous BDNF to GFP-TTC internalization by motor nerve ending, BDNF action in LAL muscles was neutralized by injecting an anti-BDNF antibody 30 min before GFP-TTC injection. Pretreating mice with 10 μg of this antibody blocked the effect of exogenous BDNF (50 ng) on GFP-TTC internalization by the NMJs of the LAL (FIG. 3). The anti-BDNF antibody pretreatment also blocked endogenous BDNF as the amount of GFP-TTC localized at nerve endings was significantly reduced by about 30%; however, pretreatment with normal chicken IgY immunoglobulin did not significantly affect localization. These results suggest that neuronal internalization of GFP-TTC utilizes a BDNF-sensitive endocytic pathway.

The blocking experiments described above were performed by injecting the mice subcutaneously with chicken anti-BDNF antibody and control mice were injected with normal chicken IgY (both at a dose of 10 μg in 50 _{I} μl of PBS) 30 minutes before injecting them (in the immediate vicinity of the LAL muscle) with GFP-TTC, or before co-injecting them with BDNF (50 ng) and GFP-TTC. GFP-TTC quantification was performed, as described above, 30 minutes post-injection of the anti-BDNF and normal antibody preparations. The experiments were performed in triplicate, and the results were expressed as the mean+/− SEM (n=20). Statistical significance was defined as P<0.05, using a two-tailed Student's t-test.

### Example 19

### Other Neurotrophic Factors Influence GFP-TTC Internalization at Motor Nerve Terminals

We determined the effect of five additional neurotrophic factors on GFP-TTC localization at the NMJ, as shown in Table 4. These neurotrophic factors included neurotrophin-3 (NT-3), NT-4 and NGF, which are members of the neurotrophin family; the neurocytokine, ciliary neurotrophic factor (CNTF), which is a member of the LIF cytokine family; and glial-derived neurotrophic factor (GDNF), which is a member of the TGF-β superfamily. Many of the effects of BDNF on neurons are mediated via the high-affinity tyrosine kinase receptor TrkB, which is also the receptor for NT-4 (Barbacid, 1995). However, a similar concentration range of NT-4 was less effective than BDNF; e.g. it elicited a maximal 1.54±0.23-fold increase of GFP-TTC localization at the NMJ, which was significantly lower than that of BDNF. The effect of GDNF was similar to that of NT-4; however, even higher concentrations of NGF, NT-3 and CNTF did not significantly affect GFP-TTC internalization.

Rat CNTF, human NT-3, human NT-4, human GDNF, and mouse NGF were purchased from Alomone Laboratories (Jerusalem, Israel). The neurotrophic factors were prepared as stock solutions (10 μg/mL), and aliquots were stored at ±80° C. until used.

### Example 20

### GFP-TTC and TrkB Receptors Localize in Adjacent Domains of Mature Mouse NMJs

Because BDNF and NT-4 increased the concentration at NMJs of GFP-TTC (Table 2), and they are known to activate TrkB (Barbacid, 1995), we examined whether GFP-TTC co-localized with TrkB at the NMJs of the LAL. Consistent with previous studies (Gonzalez et al., 1999; Sakuma et al., 2001), TrkB immunostaining was confined to the NMJ, mainly in the presynaptic side, and was adjacent to, but did not co-localize with, clusters of GFP-TTC labeling (FIG. 15). Similar results were also obtained after co-injection of BDNF and GFP-TTC. These observations suggested that the mechanism whereby BDNF influences the internalization of GFP-TTC in motor nerve terminals does not involve a direct interaction between TrkB and GFP-TTC or its receptors.

Immunostaining was performed as follows. GFP-TTC-treated (50 μg in 50 μl of PBS) and control LAL muscles were permeabilized and blocked (1 h) with PBS supplemented with 10% normal goat or rat serum, 2% bovine serum albumin and 0.02% Triton X-100. The muscle preparations subsequently were incubated overnight with rabbit polyclonal anti-TrkB antibody (1:200) or mouse monoclonal anti-NF-200 antibody (1:700) in PBS supplemented with 2% bovine albumin and 0.02% Triton X-100, followed by incubation with the appropriate Cy3-conjugated secondary antibody. LAL muscles were thus stained (45 min, 37° C.) with Alexa-Fluor 647 α-BTX (2 μg/mL PBS) and washed twice in PBS (for TrkB-labeled preparations). The LAL muscles were whole-mounted on glass slides with MOWIOL® before examination by confocal microscopy.

### Example 21

### BDNF does not Affect Spontaneous Quantal Acetylcholine Release by Mature NMJs

BDNF, NT-3, and NT-4 are known to enhance synaptic transmission between Xenopus spinal neurons and myotonal myocytes by potentiating acetylcholine release in developing neuromuscular synapses (Lohof et al., 1993; Stoop & Poo, 1996; Wang & Poo, 1997). This observation suggested that the increased internalization and concentration of GFP-TTC in motor nerve endings that is mediated by BDNF may be due to BDNF-induced changes in the exocytosis or endocytosis quantal transmitter release, which affects the synaptic vesicle recycling pathway. To test this possibility, the acute effect of BDNF on synaptic function was examined by determining its possible effect on spontaneous quantal acetylcholine release. Isolated LAL muscle-nerve preparations were mounted in a recording chamber, and intracellular synaptic potentials were recorded before (control) and after bath application of BDNF (250 ng/mL). BDNF did not significantly affect the frequency of MEPPS (normalized MEPP frequency: 1.029±0.03). However, in order to eliminate a possible effect of GFP-TTC, similar experiments were performed with GFP-TTC (250 ng/mL)-treated preparations. MEPPs were measured during a 45-min recording period; however, as before, their frequency was not altered (normalized MEPP frequency: 1.034±0.004). These results indicate that enhancement of GFP-TTC's internalization by mature NMJs by BDNF is not caused by its potentiation of spontaneous quantal acetylcholine release.

The electrophysiological analysis described above was performed by isolating LAL muscles with their associated nerves, from female Swiss mice (20-30 g) killed by dislocation of their cervical vertebrae. The muscles were dissected and mounted in Rhodorsil®-lined organ baths superfused at room temperature (20-22° C.) with a standard, oxygenated, physiological Ringer's solution of the following composition (in mM): NaCl, 154; KCl, 5; CaCl₂, 2; MgCl₂, 1; HEPES buffer, 5 (pH 7.4); and glucose, 11. Intracellular recordings of the miniature endplate potential (MEPP) of surface fibers were made with micro-electrodes filled with 3 M KCl solution (resistance=8-15 MΩ). using conventional techniques and an Axoclamp-2A system (Axon Instruments, Union City, Calif., USA). Recordings were made randomly from 6-10 different endplates before (control) and for 30-45 minutes after treatment with BDNF (250 ng/ml) or GFP-TTC (100 μg/ml). The MEPP frequency values were normalized with respect to control values, and the means+/− SD of three independent experiments were determined.

TABLE 4
Effect of various neurotrophic factors on GFP-mediated
fluorescence of nerve terminals after co-injecting mice with
GFP-TTC and the individual factors

| | | Mean |
|---|---|---|
| | Neurotrophic factor | relative increase in |
| Neurotrophic factor | receptor | fluorescence |
| BDNF | TrkB | 2.12 ± 0.19** |
| NT-4 | TrkB | 1.49 ± 0.23** |
| NT-3 | TrkC | 0.94 ± 0.05 |
| NGF | TrkA | 1.06 ± 0.06 |
| CNTF | CNTFRα | 0.95 ± 0.05 |
| GDNF | GFRα/c-RET | 1.51 ± 0.02* |

GFP-mediated fluorescence was quantified, by confocal microscopy, 30 min after co-injecting LAL muscles with GFP-TTC and various amounts of the neurotrophic factors. The means of the relative increases in fluorescence were determined from two or three independent experiments. Maximal increases in fluorescence were elicited with 50 ng of the neurotrophic factors, except for 2.5 ng of NT-3.
**P = 0.005;
*P = 0.05.

### Example 22

### BDNF does not Increase Retrograde Axonal Transport of 3-gal-TTC

Significant axonal GFP labeling was detected after GFP-TTC injected with the higher doses of BDNF (FIG. 13A, arrows). Therefore, in order to determine whether BDNF affected the fusion protein's retrograde axonal transport, β-gal activity was compared in the nerve region under the ligature (the B-segment) one hour after β-gal-TTC i.m. injection into the gastrocnemius muscle (FIG. 16 and Table 5). Under control conditions, only weak β-gal activity was detected in the nerve's B-segment, and co-injection of BDNF and β-gal-TTC significantly increased the B-segment's activity. Comparing the percentages of internalized and transported β-gal activity (Tables 3 and 5) revealed a similar effect of BDNF, indicating that the enhancement of enzymatic activity in the B segment occurs as a consequence of the increased amount internalized rather than a direct effect of BDNF on the retrograde transport of β-gal-TTC.

TABLE 5
The effect of BDNF on retrograde transport of β-gal-TTC by the
sciatic nerve

| | | Transported activity/ |
|---|---|---|
| | β-gal activity transported (IU) | internalized activity (%) |
| Control | 517.835 ± 175.706 | 5.18 ± 1.08% |
| BDNF 5 ng | 1565.169 ± 43.994 | 8.86 ± 0.81% |
| BDNF 50 ng | 2602.057 ± 271.254 | 14.18 ± 1.24% |

Values of β-gal activity retrogradely transported and thus detected under the ligature (B-segment only) represent the mean ± SD of three independent experiments. This activity was also expressed as percentage of internalized activity. Each value represents the mean retrograde transported β-gal activity (the activity in the B-segment) expressed as a percentage of the internalized β-gal activity. Each value represents the mean ± SD of three independent experiments.

### REFERENCES

The following publications, which have been cited herein, are relied upon and incorporated in their entireties herein.
Eisel, U., Jarausch, W., Goretzki, K., Henschen, A., Engels, J., Weller, U., Hudel, M., Habermann, E. & Niemann, H. (1986) EMBO J. 5, 2495-2502.
Fairweather, N. F. & Lyness, V. A. (1986) Nucleic Acids Res. 14, 7809-7812.
Montecucco, O. & Schiavo, G. (1995) Quart. Rev. Biophys. 28, 423-472.
Schwab, M. E. & Thoenen, H. (1976) Brain Res. 105, 213-227.
Schwab, M. E. & Thoenen, H. (1977) Brain Res. 122, 459-474.
Price, D. L., Griffin, J. W. & Peck. K. (1977) Brain Res. 121, 379-384.
Bizzini, B., Stoeckel, K. & Schwab, M. (1977) J. Neurochem. 28, 529-542.
Evinger, O. & Erichsen, J. T. (1986) Brain Res. 380, 383-388.
Fishman, P. S. & Carrigan, D. R. (1987) Brain Res. 406, 275-279.
Manning, K. A., Erichsen, J. T. & Evinger, O. (1990) Neurosci. 34, 251-263.
Halpern, J. L., Habig, W. H., Neale, E. A. & Stibitz, S. (1990) Infection and Immunity 58, 1004-1009.
Bizzini, B., Grob, P., Glicksman, M. A. & Akert, K. (1980) Brain Res. 193, 221-227.
Dobrenis, K., Joseph, A. & Rattazzi, M. G. (1992) Proc. Natl. Acad. ScL USA 89, 2297-2301.
Fishman, P. S. & Savitt, J. M. (1989) Exp. Neurol. 106, 197-203.
Beaude, P., Delacour, A., Bizzini, B., Domuado, D. & Remy, M. H. (1990) Biochem. 271, 87-91.
Francis, J. W., Hosier, B. H., Brown, R. H., Jr. & Fishman, P. S. (1995) J. Biol. Chem. 270, 15434-15442.
Fellous. M., Gunther, E., Kemler, R., Weils, J., Berger, R., Guenet, J. L., Jakob, H. & Jacob, F. (1978) J. Exp. Med. 148, 58-70.
Jakob, H. & Nicolas, J. F. (1987) Methods Enzymol. 151, 66-84.
Whitehouse, R. L. S., Benichou, J. O. & Ryter, A. (1977) Biol. Cell. 30, 155-158.
Wojcik, B. E., Nothias, F., Lazar, M., Jouin, H., Nicolas, J. F. & Peschanski, M. (1993) Proc. Natl. Acad. ScL USA 90, 1305-1309.
Price, D. L., Griffin, J. Young, A. Peck, K. & Stocks, A. (1975) Science 188, 945-947.
Matteoli, M., Verderio, C., Rossetto, O., lezzi, N., Ooco. S. Schiavo, G. & Montecucco, G, (1996) Proc. Natl. Acad ScL USA 93, 13310-13315.
Stockel, K., Schwab, M. & Thoenen, H. (1975) Brain Res. 99, 1-16.
Ugolini, G. (1995a) in Viral Vectors: Gene Therapy and Neuroscience Applications, eds. Loewy, A. D. & Kaplitt M. G. (New York: Academic Press), pp. 293-317.
Ugolini, G. (1995b) The Journal of Comparative Neurology 356, 457-480.
Borke, R. C., Nau, M. E. & R. L Ringler, J. (1983) Brain Research 269, 47-55.
Horst, G. T. T., Copray, J. C. V. M., liem, R. S. B. & Willigen, J. D. V. (1991) Neuroscience 40, 735-758.
O′Reilly, P. M. R. & Fitzgerald, M. J. T. (1990) J. Anat. 172, 227-243.
Chalfie, M. Tu, Y., Euskirchen, G., Ward, W. W. & Prasher, D. C. (1994) Science 263, 802-805.
Miesenbock, G. & Rothman, J. E. (1997) Proc. Natl. Acad. Sci USA 94, 3402-3407.
Le Mouellic, H., Lallemand, Y. & Brûlet, P. (1990) Proc. Natl. Acad. Sci USA 87, 4712-4716.
Le Mouellic, H., Lallemand, Y. & Brûlet, P. (1992) Cell 69, 251-264.
Mombaerts, P., Wang, F., Dulac, C. Chao, S. K., Nemes, A., Mendelsohn, M., Edmondson, J. & Axel, R. (1996) Cell 87, 675-686.
Mountford, P. S. & Smith, A. G. (1995) Trends Genet. 11, 179-184.
Rosen, D. R. & al (1993) Nature 362, 59-62.
Lefebvre, S., Büglen, L., Reboullet, S. Clermont, O., Bûrlet, P., Viollet, L, Benichou, B., Cruaud, O., Millasseau, P., Zeviani, M., Paslier, D. L., Frézal, J., Cohen, D., Weissenbach, J., Munnich, A. & Melki, J. (1995) Cell 80, 155-165.
Roy, N. & al (1995) Cell 80, 167-178.
Wolfe, J. H., Deshmane, S. L. & Fraser, N. W. (1992) Nature genetics 1, 379-384.
Sango, K., Yamanaka, S., Hoffmann, A., Okuda, Y., Grinberg, A., Westphal, H., McDonald, M. P., Crawley, J. N., Sandhoff, K., Suzuki, K. & Proia, R. L. (1995) Nature Genetics 11, 170-176.
Duarte, R. G. (1995) Neurologia 1, 56-61.
Ghadge, G. D., Roos, R. P., Kang, U. J., Wollmann, R., Fishman, P. S., Kalynych, A M., Barr, E. & Leiden, J. M. (1995) Gene Therapy 2, 132-137.
Anderson, R. O. W. 1998. The caveolae membrane system. Annu. Rev. Biochem. 67:199-225.
Bothwell, M. (1995) Functional interactions of neurotrophins and neurotrophin receptors. Annu. Rev. Neurosci., 18, 223-225.
Airaksinen, M. S. & Saarma, M. (2002) The GDNF family: signaling, biological functions and therapeutic value. Nat. Rev. Neurosci., 3, 383-394.
Barbacid, M. (1995) Neurotrophic factors and their receptors. Curr. Opin. Neurobiol., 7, 148-155.
Bhattacharyya, A., Watson, F. L., Pomeroy, S. L., Zhang, Y. Z., Stiles, C. D. & Segal, R. A. (2002) High-resolution imaging demonstrates dynein-based vesicular transport of activated Trk receptors. J. Neurobiol., 51, 302-312.
Boulanger, L. & Poo, M. M. (1999) Gating of BDNF-induced synaptic potentiation by cAMP. Science, 284, 1982-1984.
Bronfman, F. C., Tcherpakov, M., Jovin, T. M. & Fainzilber, M. (2003) Ligand-induced internalization of the p75 neurotrophin receptor: a slow route to the signaling endosome. J. Neurosci., 23, 3209-3220.
Chaïb-Oukadour, I., Gil, C. & Aguilera, J. (2004) The C-terminal domain of the heavy chain of tetanus toxin rescues cerebellar granule neurones from apoptotic death: involvement of phosphatidylinositol 3-kinase and mitogen-activated protein kinase pathways. J. Neurochem., 90, 1227-1236.
Coen, L., Osta, R., Maury, M. & Brûlet, P. (1997) Construction of hybrid proteins that migrate retrogradely and transynaptically into the central nervous system. Proc. Natl. Acad. Sci. USA, 94, 9400-9405.
Dechant, G. & Barde, Y. A. (1997) Signaling through the neurotrophin receptor p75NTR. Curr. Opin. Neurobiol., 7, 413-418.
Du, J., Feng, L., Yang, F. & Lu, B. (2000) Activity and Ca²⁺-dependent modulation of surface expression of brain-derived neurotrophic factor receptors in hippocampal neurons. J. Cell Biol., 150, 1423-1434.
Duchemin, A.-M., Ren, Q., Mo, L., Neff, N. H. & Hadjiconstantinou, M. (2002) GM1 ganglioside induces phosphorylation and activation of Trk and Erk in brain. J. Neurochem., 81, 696-707.
Durbec, P., Marcos-Gutierrez, C. V., Kilkenny, C., Grigoriou, M., Wartiowaara, K., Suvanto, P., Smith, D., Ponder, B., Costantini, F., Saarma, M., Sariola, H. & Pachnis, V. (1996) GDNF signalling through the Ret receptor tyrosine kinase. Nature, 381, 789-793.
Ehlers, M. D., Kaplan, D. R., Price, D. J. & Koliatsos, V. E. (1995) NGF-stimulated retrograde transport of trkA in the mammalian nervous system. J. Cell Biol., 130, 149-156.
Fan, G., Egles, C., Sun, Y., Minichiello, L., Renger, J. J., Klein, R., Liu, G. & Jaenisch, R. (2000) Knocking the NT4 gene into the BDNF locus rescues BDNF deficient mice and reveals distinct NT4 and BDNF activities. Nat. Neurosci., 3, 350-357.
Forlani, S. & Nicolas, J. F. (1996) Quantification of minute levels of βgalactosidase in individual 2- to 16-stage mouse embryos. Trends Genet., 12, 498-500.
Funakoshi, H., Belluardo, N., Arenas, E., Yamamoto, Y., Casabona, A., Persson, H. & Ibanez, C. F. (1995) Muscle-derived neurotrophin-4 as an activity-dependent trophic signal for adult motor neurons. Science, 268, 1495-1499.
Funakoshi, H., Frisen, J., Barbany, G., Timmusk, T., Zachrisson, O., Verge, V. M. K. & Persson, H. (1993) Differential expression of mRNAs for neurotrophins and their receptors after axotomy of the sciatic nerve. J. Cell Biol., 123, 455-465.
Galbiati, F., Razani, B. & Lisanti, M. P. (2001) Emerging themes in lipid rafts and caveolae. Cell, 106, 403-411.
Gil, C., Chaib, I., Blasi, J. & Aguilera, J. (2001) Hc fragment of tetanus toxin activates protein kinase C isoforms and phosphoproteins involved in signal transduction. Biochem. J., 356, 97-103.
Gil, C., Chaib, I., Pellicioni, P. & Aguilera, J. (2000) Activation of signal transduction pathways involving TrkA, PLCγ, PKC isoforms and ERK-1/2 by tetanus toxin. FEBS Lett., 481, 177-182.
Gil, C., Chaib-Oukadour, I. & Aguilera, J. (2003) C-terminal fragment of tetanus toxin heavy chain activates Akt and MEK/ERK signaling pathways in a Trk receptor-dependent manner in cultured cortical neurons. Biochem. J., 373, 613-620.
Gomez-Pinilla, F., Ying, Z., Roy, R. R. & Edgerton, V. R. (2001) Differential regulation by exercise of BDNF and NT-3 in rat spinal cord and skeletal muscle. Eur. J. Neurosci., 13, 1078-1084.
Gonzalez, M., Ruggiero, F. P., Chang, Q., Shi, Y.-J., Rich, M. M., Kraner, S. & Balice-Gordon, R. J. (1999) Disruption of TrkB-mediated signaling induces disassembly of postsynaptic receptor clusters at neuromuscular junction. Neuron, 24, 567-583.
Halpern, J. L. & Neale, E. A. (1995) Neurospecific binding, internalization, and retrograde axonal transport. Curr. Top. Microbiol. Immunol., 195, 221-241.
Herreros, J., Ng, T. & Schiavo, G. (2001) Lipid rafts act as specialized domains for tetanus toxin binding and internalization into neurons. Mol. Biol. Cell, 12, 2947-2960.
Higuchi, H., Yamashita, T., Yoshikawa, H. & Tohyama, M. (2003) PKA phosphorylates the p75 receptor and regulates its localization to lipid rafts. EMBO J., 22, 1790-1800.
Huang, E. J. & Reichardt, L. F. (2003) Trk receptors: roles in neuronal signal transduction. Annu. Rev. Biochem., 72, 609-642.
Ip, N. Y., Stitt, T. N., Tapley, P., Klein, R., Glass, D. J. & Fandl, J. (1993) Similarities and differences in the way neurotrophins interact with the trk receptors in neuronal and normeuronal cells. Neuron, 10, 137-149.
Kissa, K., Mordelet, E., Soudais, C., Kremer, E. J., Demeneix, B. A., Brûlet, P. & Coen, L. (2002) In vivo neuronal tracing with GFPTTC gene delivery. Mol. Cell. Neurosci., 20, 627-637.
Klein, R., Smeyne, R. J., Wurst, W., Long, L. K. A. A. B., Joyner, A. L. & Barbacid, M. (1993) Targeted disruption of the trkB neurotrophin receptor gene results in nervous system lesions and neonatal death. Cell, 75, 113-122.
Koliatsos, V. E., Cayouette, M. H., Berkemeier, L. R., Clatterbuck, R. E., Price, D. J. & Rosenthal, A. (1994) Neurotrophin 4/5 is a trophic factor for mammalian facial motor neurons. Proc. Natl. Acad. Sci USA, 91, 3304-3308.
Lalli, G. & Schiavo, G. (2002) Analysis of retrograde transport in motor neurons reveals common endocytic carriers for tetanus toxin and neurotrophin receptor p75^{NTR.} J. Cell Biol., 156, 233-239.
Liou, J. C., Yang, R. S. & Fu, W. M. (1997) Regulation of quantal secretion by neurotrophic factors at developing motoneurons in Xenopus cell cultures. J. Physiol., 503, 129-139.
Liu, N. Q., Lossinsky, A. S., Popik, W., Li, X., Gujuluva, C., Kriederman, B., Rpberts, J., Pushkarsky, T., Bukrinsky, M., Witte, M., Weinand, M. & Fiala, M. (2002) Human immunodeficiency virus type I enters brain microvascular endothelia by macropinocytosis dependent on lipid rafts and the mitogenactivated protein kinase pathway. J. Virol., 76, 6689-6700.
Lohof, A. M., Ip, N. Y. & Poo, M. M. (1993) Potentiation of developing neuromuscular synapses by the neurotrophins NT-3 and BDNF. Nature, 363, 350-353.
Maskos, U., Kissa, K., Saint Cloment, C. & Brûlet, P. (2002) Retrograde transsynaptic transfer of green fluorescent protein allows the genetic mapping of neuronal circuits in transgenic mice. Proc. Natl. Acad. Sci. USA, 99, 10120-10125.
Mendell, L. M., Johnson, R. D. & Munson, J. B. (1999) Neurotrophin modulation of the monosynaptic reflex after peripheral nerve transection. J. Neurosci., 19, 3162-3170.
Meyer-Franke, A., Wilkinson, G. A., Kruttgen, A., Hu, M., Munro, E., Hanson, M. G. J., Reichardt, L. F. & Barres, B. A. (1998) Depolarization and cAMP elevation rapidly recruit TrkB to the plasma membrane of CNS neurons. Neuron, 21, 681-693.
Miana-Mena, F. J., Munoz, M. J., Ciriza, J., Soria, J., Brûlet, P., Zaragoza, P. & Osta, R. (2003) Fragment C tetanus toxin: a putative activity-dependent neuroanatomical tracer. Acta Neurobiol. Exp., 63, 211-218.
Miana-Mena, F. J., Munoz, M. J., Roux, S., Ciriza, J., Zaragoza, P., Brûlet, P. & Osta, R. (2004) A non-viral vector for targeting gene therapy to motoneurons in the CNS. Neurodegenerative Dis., 1, 101-108.
Miana-Mena, F. J., Roux, S., Benichou, J.-C., Osta, R. & Brûlet, P. (2002) Neuronal activity-dependent membrane traffic at the neuromuscular junction. Proc. Natl. Acad. Sci USA, 99, 3234-3239.
Munson, J. B., Johnson, R. D. & Mendell, L. M. (1997) NT-3 increases amplitude of EPSPs produced by axotomized group Ia afferents. J. Neurophysiol., 77, 2209-2212.
Paratcha, G., Ledda, F., Baars, L., Coulpier, M., Besset, V., Anders, J., Scott, R. & Ibanez, C. F. (2001) Released GFRalpha 1 potentiates downstream signaling, neuronal survival, and differentiation via a novel mechanism of recruitment of c-Ret to lipid rafts. Neuron, 29, 171-184.
Pitto, M., Mutoh, T., Kuriyama, M., Ferraretto, A., Palestini, P. & Masserini, M. (1998) Influence of endogenous GM1 ganglioside on TrkB activity in cultured neurons. FEBS Lett., 439, 93-96.
Ribchester, R. R., Thomson, D., Haddow, L. J. & Ushkaryov, Y. A. (1998) Enhancement of spontaneous transmitter release at neonatal mouse neuromuscular junctions by the glial cell line-derived neurotrophic factor (GDNF). J. Physiol., 512, 635-641.
Rind, H. B., Butowt, R. & von Bartheld, C. S. (2005) Synaptic targeting of retrogradely transported trophic factors in motoneurons: comparison of Glial cell line-derived neurotrophic factor, brain-derived neurotrophic factor, and cardiotrophin-1 with tetanus toxin. J. Neurosci., 25, 539-549.
Roux, S., Colasante, C., Saint Cloment, C., Barbier, J., Curie, T., Girard, E., Molgó, J. & Brûlet, P. (2005) Internalization of GFP-tetanus toxin C-terminal fragment fusion protein at mature neuromuscular junctions. Mol. Cell. Neurosci., 30, 79-89.
Sagot, Y., Rossé, T., Vejsada, R., Perrelet, D. & Kato, A. C. (1998) Differential effects of neurotrophic factors on motoneuron retrograde labeling in a murine model of motoneuron disease. J. Neurosci., 18, 1132-1141.
Sakuma, K., Watanabe, K., Sano, M., Uramoto, I., Nakano, H., Li, Y.-J., Kaneda, S., Sorimachi, Y., Yoshimoto, K., Yasuhara, M. & Totsuka, T. (2001) A possible role for BDNF, NT-4 and TrkB in the spinal cord and muscle of rat subjected to mechanical overload, bupivacaine injection and axotomy. Brain Res., 907, 1-19.
Sakurai, T., Nagata, R., Yamanaka, A., Kawamura, H., Tsujino, N., Muraki, Y., Kageyama, H., Kunita, S., Takahashi, S., Goto, K., Koyama, Y., Shioda, S. & Yanagisawa, M. (2005) Input of orexin/hypocretin neurons revealed by a genetically encoded tracer in mice. Neuron, 46, 297-308.
Sapir, T., Geiman, E. J., Wang, Z., Velasquez, T., Mitsui, S., Yoshihara, Y., Frank, E., Alvarez, F. J. & Goulding, M. (2004) Pax6 and Engrailed1 regulate two distinct aspects of Renshaw cell development. J. Neurosci., 24, 1255-1264.
Seebach, B. S., Arvanov, V. & Mendell, L. M. (1999) Effects of BDNF and NT-3 on development of Ia/motoneuron functional connectivity in neonatal rats. J. Physiol., 81, 2398-2405.
Segal, R. A. & Greenberg, M. E. (1996) Intracellular signaling pathways activated by neurotrophic factors. Annu. Rev. Neurosci., 19, 463-469.
Silos-Santiago, I., Fagan, A. M., Garber, M., Fritzsch, B. & Barbacid, M. (1997) Severe sensory deficits but normal CNS development in newborn mice lacking TrkB and TrkC tyrosine protein kinase receptors. Eur. J. Neurosci., 9, 2045-2056.
Simons, K. & Ikonen, E. (1997) Functional rafts in cell membranes. Nature, 387, 569-572.
Simons, K. & Toomre, D. (2000) Lipid rafts and signal transduction. Nat. Rev. Mol. Cell. Biol., 1, 31-39.
Stoop, R. & Poo, M. M. (1996) Synaptic modulation by neurotrophic factors: differential and synergistic effects of brain-derived neurotrophic factor and ciliary neurotrophic factor. J. Neurosci., 16, 3256-3264.
Takahashi, M. (2001) The GDNF/RET signaling pathway and human diseases. Cytokine Growth Factor Rev., 12, 361-373.
Tang, P., Sutherland, C. L., Gold, M. R. & Finlay, B. B. (1998) Listeria monocytogenes invasion of epithelial cells requires the MEK-1/ERK-2 mitogen-activated protein kinase pathway. Infect. Immun., 66, 1106-1112.
Tansey, M., Baloh, R. H., Milbrandt, J. & Johnson, E. M. J. (2000) GFRa mediated localization of RET to lipid rafts is required for effective downstream signaling, differentiation and neuronal survival. Neuron, 25, 611-623.
Trupp, M., Arenas, E., Fainzilber, M., Nilsson, A. S., Sieber, B. A., Grigoriou, M., Kilkenny, C., Salazar-Gruesco, E., Pachnis, V., Arumae, U., Sariola, H., Saarma, M. &
Ibañez, C. F. (1996) Functional receptor for GDNF encoded by the c-ret proto-oncogene. Nature, 381, 785-789.
Tsui-Pierchala, B. A., Encinas, M., Milbrandt, J. & Johnson, E. M. J. (2002) Lipid raft in neuronal signaling and function. Trends Neurosci., 25, 412-417.
Valdez, G., Akmentin, W., Philippidou, P., Kuruvilla, R., Ginty, D. D. & Halegoua, S. (2005) Pincher-mediated macroendocytosis underlies retrograde signaling by neurotrophin receptors. J. Neurosci., 25, 5236-5247.
Vyas, K. A., Patel, H. V., Vyas, A. A. & Schnaar, R. L. (2001) Segregation of gangliosides GM1 and GD3 on cell membranes, isolated membrane rafts, and defined supported lipid monolayers. Biol. Chem., 382, 241-250.
Wang, X. H. & Poo, M. M. (1997) Potentiation of developing synapses by postsynaptic release of neurotrophin-4. Neuron, 19, 825-835.
Wu, C., Butz, S., Ying, Y. & Anderson, R. G. W. (1997) Tyrosine kinase receptors concentrated in caveolae-like domains from neuronal plasma membrane. J. Biol. Chem., 272, 3554-3559.
Xie, K., Wang, T., Olafsson, P., Mizuno, K. & Lu, B. (1997) Activitydependent expression of NT-3 in muscle cells in culture: implications in the development of neuromuscular junctions. J. Neurosci., 17, 2947-2958.
Yan, Q., Elliott, J. L., Matheson, C., Sun, J., Zhang, L., Mu, X., Rex, K. L. & Snider, W. D. (1993) Influences of neurotrophins on mammalian motoneurons in vivo. J. Neurobiol., 24, 1555-1577.

1-67. (canceled)

68. A method of modulating the transport in a neuron of a tetanus toxin or a fusion protein comprising a fragment C of the tetanus toxin, wherein the method comprises administering to the neuron a TrkB receptor antagonist in an amount sufficient to modulate the neuronal transport of the tetanus toxin or the fusion protein.

69. The method according to claim 68, wherein the TrkB receptor antagonist decreases the internalization of the tetanus toxin or fusion protein at a neuromuscular junction.

70. The method according to claim 69, wherein the TrkB receptor antagonist is an antibody that binds to a TrkB receptor agonist, thereby reducing activation of a TrkB receptor.

71. The method according to claim 70, wherein the TrkB receptor agonist is a neurotrophic factor that activates a TrkB receptor.

72. The method according to claim 71, wherein the neurotrophic factor is a Brain Derived Neuotrophic Factor (BDNF) or a NT-4.

73. The method according to claim 72, wherein the neurotrophic factor is BDNF.

74. The method according to claim 73, wherein the internalization of the tetanus toxin fusion protein at the neuromuscular junction is decreased.

75. The method according to claim 70, wherein the antagonist is administered concurrently with the tetanus toxin or a fusion protein.

76. A method of modulating the transport in a neuron of a tetanus toxin or a fusion protein comprising a fragment C of the tetanus toxin, wherein the method comprises administering to the neuron a GFRα/cRET receptor antagonist in an amount sufficient to modulate the neuronal transport of the tetanus toxin or the fusion protein.

77. The method according to claim 76, wherein the GFRα/cRET receptor antagonist decreases the internalization of the tetanus toxin or fusion protein at a neuromuscular junction.

78. The method according to claim 77, wherein the GFRα/cRET receptor antagonist is an antibody that binds to a GFRα/cRET receptor agonist, thereby reducing activation of a GFRα/cRET receptor.

79. The method according to claim 78, wherein the GFRα/cRET receptor agonist is a neurotrophic factor that activates a GFRα/cRET receptor.

80. The method according to claim 79, wherein the neurotrophic factor is a GDNF.

81. The method of claim 80, wherein the internalization of the tetanus toxin at the neuromuscular junction is decreased.

82. The method according to claim 78, wherein the neurotrophic factor is administered concurrently with the tetanus toxin fusion protein.
